(19) United States
(12) Patent Application Publication
(10) Pub. No.: US 2005/0119739 A1
(43) Pub. Date: Jun. 2, 2005

(54) Multi-focal intraocular lens, and methods for making and using same

(75) Inventor: Alan N. Glazier, Rockville, MD (US)

Correspondence Address: David S. Taylor;Liniak, Berenato & White, LLC, Suite 240, 6550 Rock Spring Dr., Bethesda, MD, 20817, US

(73) Assignee: Vision Solution Technologies, LLC

(21) Appl. No.: 11/007,345

(22) Filed: Dec. 9, 2004

Related U.S. Application Data
(63) Continuation-in-part of application No. 10/733,173, filed on Dec. 10, 2003.
Application No. 10/733,173 is a continuation-in-part of application No. PCT/US02/17964, filed on Jun. 7, 2002.
Application No. PCT/US02/17964 is a continuation-in-part of application No. 10/158,574, filed on May 30, 2002, now Pat. No. 6,855,164.
Application No. 10/158,574 is a continuation-in-part of application No. 10/139,144, filed on May 3, 2002, now abandoned.
(60) Provisional application No. 60/297,306, filed on Jun. 11, 2001.

(51) Int. Cl. A61F 2/16

(52) U.S. Cl. 623/6.13; 623/6.37; 623/6.27

(57) ABSTRACT

An intraocular lens and related method for optically altering an image peripheral to a scotomatous area in a visual field of a person having a retinal degenerative condition are provided. Also provided are an intraocular lens and related method for reducing the effect of a scotomous area in a visual field. The intraocular lens includes first and second fluids in an optic body.

### CROSS-REFERENCE TO RELATED APPLICATIONS

This application is a continuation in part of U.S. patent application Ser. No. 10/733,173, filed Dec. 10, 2003, which is a continuation in part of PCT/US02/17964, filed Jun. 7, 2002, which is a continuation-in-part of U.S. patent application Ser. No. 10/158,574, filed May 30, 2002, which is a continuation-in-part of U.S. patent application Ser. No. 10/139,144, filed May 3, 2002 (now abandoned), the complete disclosures of which are incorporated herein by reference.

This application claims the benefit of priority of provisional patent application 60/297,306 filed Jun. 11, 2001, the complete disclosure of which is incorporated herein by reference.

### BACKGROUND OF THE INVENTION

1. Field of the Invention

This invention relates generally to the use of intraocular lenses for treatment of retinal degenerative conditions (e.g., low vision, macular degeneration), and other conditions and surgical procedures involving the eyes, especially human eyes.

2. Description of Related Art

Generally, the most outwardly visible structures of the human eye include an optically clear anterior cornea, the iris sphincter sitting behind the cornea, and the aperture of the iris, which aperture is referred to as the pupil. The pupil appears as a circular opening concentrically inward of the iris. Light passes through the pupil along a path to the retina in the back of the eye. In a healthy human eye, a physiological crystalline lens in a capsular bag is positioned posterior to the iris. The chamber between the posterior cornea and the front surface of the capsular bag is commonly referred to in the art as the anterior chamber. A posterior chamber is the area behind the anterior chamber, and includes the capsular bag and physiological crystalline lens.

Ciliary muscle concentrically surrounds the capsular bag, and is coupled to the physiological crystalline lens by suspensory ligaments, also known as zonules. Vitreous humor is contained in the posterior chamber behind the capsular bag. The vitreous humor is surrounded by the retina, which is surrounded by the sclera. The functions and interrelationship of these structures of the human eye are well known in the art and, for this reason, are not elaborated upon in detail herein, except as is needed or useful for facilitating an understanding of this invention.

Light entering the emmetropic human eye is converged towards a point focus on the retina at a point known as the fovea. The cornea and tear film are responsible for the initial convergence of entering light. Subsequent to refraction by the cornea, the light passes through the physiological crystalline lens, where the light is refracted again. When focusing on an object, ideally the physiological crystalline lens refracts incoming light towards a point image on the fovea of the retina. The amount of bending to which the light is subjected is termed the refractive power. The refractive power needed to focus on an object depends upon how far away the object is from the principle planes of the eye. More refractive power is required for converging light rays to view close objects clearly than is required for converging light rays to view distant objects clearly.

A young and healthy physiological lens of the human eye has sufficient elasticity to provide the eye with natural accommodation ability. A young elastic lens may alter its shape, by a process known as accommodation, to change refractive power. The term accommodation refers to the ability of the eye to adjust focus between the distant point of focus, called the Punctum Remotum orpr (far point beyond 20 feet or 6 meters away), and the near point of focus called the Punctum Proximum orpp (near point within 20 feet or 6 meters away from the eye). Focus adjustment is performed in a young elastic lens using the accommodative-convergence mechanism. The ciliary muscle functions to shape the curvature of the physiological crystalline lens to an appropriate optical configuration for focusing and converging light rays entering the eye on the fovea of the retina. It is widely believed that this accommodation is accomplished via contracting and relaxing the ciliary muscle, which accommodate the lens of the eye for near and distant vision, respectively.

A retinal degenerative conditions (RDC) adversely affects an individual's eyesight, both at near and far vision. Generally, an RDC involves damage to the fovea. An RDC such as macular degeneration leaves the afflicted individual with “blind spot” or scotoma usually at or near the center of a person's visual field. The afflicted individual is often only able to see peripheral images around the blind spot. The visual field provided by such peripheral images is often insufficient to allow the individual to perform routine activities such as reading, driving a vehicle, or even daily chores and errands. For example, when an individual having a RDC attempts to recognize another person at a distance, the individual may be able to discern the eccentric body portions of the viewed person peripherally, but the scotoma may “wipe out” the facial details of the viewed person, rendering the person unrecognizable.

The person who suffers from RDC's is typically treated optically by using magnification or prism in lens form. At distance, a Galilean telescopic magnifying device may be placed in front of the eye or in the eye and customized to the user's needs. The magnification of the device enlarges the image viewed, expanding the image into more healthy areas of retina peripheral (eccentric) to the scotoma. At near, the person suffering from a RDC usually needs magnification in the form of magnifying plus powered lenses and/or prisms—the former (i.e., the plus lenses and magnifiers) to help enlarge the image outside of the scotoma as in the telescopic example and the latter (e.g., the prisms) to help shift the images to different, more functional areas of the retina.

Devices used to provide magnification at distance and near are prescribed according to the art and science of “low-vision”. An example of a low vision device for distance use is a spectacle-mounted telescopic device. An example of a low vision device for near use is a hand-held magnification device and/or prism to assist the user in using retinal area peripheral to the damaged area responsible for producing the scotoma. Devices used to provide magnification at distance and near have several drawbacks. First of all, the devices are heavy and bulky, making them difficult to use from an ergonomic perspective. Second, the devices, such as those mounted on a pair of spectacles, may be considered aesthetically unappealing by some. Third, the devices may distort (e.g., create aberrations, astigmatism) or decrease the effectiveness of magnification, for example, in the case of spectacle-mounted telescopic devices in which there exists a vertex distance (the distance from the back of the lens to the front of the cornea). Fourth, current implantable telescopic lenses are held within bulky housings, which decrease the user's peripheral vision and result in a significant loss in the user's field of vision. Fifth, in the example of near vision magnification, the devices are often housed in a hand held device, which requires the user to not have “hands free” use of the device—i.e., the user may have trouble holding a newspaper in one hand and a device in the other.

### OBJECTS OF THE INVENTION

An object of this invention is to provide an intraocular lens (IOL) useful for reducing the effects of a scotomatous area in a field of vision of a person having a retinal degenerative disorder.

It is another object of this invention to provide a method for reducing the effects of a scotomatous area in a field of vision of a person having a retinal degenerative disorder.

Another object of this invention is to provide an IOL that reduces the adverse effects of RDCs while permitting the user to shift between near and far vision by natural tilting movement of the head and/or eye, preferably smoothly and without significant disruption to the field of vision.

Additional objects and advantages of the invention will be set forth in the description that follows, and in part will be apparent from the description, or may be learned by practice of the invention. The objects and advantages of the invention may be realized and obtained by means of the instrumentalities and combinations pointed out in the appended claims.

### SUMMARY OF THE INVENTION

To achieve one or more of the foregoing objects, and in accordance with the purposes of the invention as embodied and broadly described herein, an aspect of the invention provides a method for optically altering an image peripheral to a scotomatous area in a visual field of a person having a retinal degenerative condition. The method comprises inserting an ocular lens into an eye of a person having a retinal degenerative condition characterized by a scotomatous area in a visual field, the ocular lens comprising an optic body, an optically transmissive primary fluid, and an optically transmissive secondary fluid. The optic body comprises an anterior wall, a posterior wall, and a chamber between the anterior wall and the posterior wall. The optically transmissive primary and secondary fluids are contained in the chamber and have different densities and refractive indexes from one another.

In accordance with the purposes of this invention as embodied and broadly described herein, another aspect of the invention provides a method for optically altering an image peripheral to a scotomatous area in a visual field of a person having a retinal degenerative condition. The method of this aspect of the invention comprises inserting an ocular lens into an eye of a person having a retinal degenerative condition characterized by a scotomatous area in a visual field, the ocular lens comprising an optic body, an optically transmissive primary fluid, and an optically transmissive secondary fluid. The optic body comprises an anterior wall, a posterior wall, and a chamber between the anterior wall and the posterior wall. The optically transmissive primary and secondary fluids are contained in the chamber and have different densities and refractive indexes from one another. The ocular lens has a first power in straight ahead gaze and a second power in down gaze. An objective lens having a third power is provided in front of the ocular lens to establish a telescopic effect. Orienting the human eye in a generally straight ahead gaze for far vision passes the visual axis through the primary liquid, but not the secondary liquid, for focusing on a distant point. Movement of the eye into a downward gaze passes the visual axis through the primary fluid and the secondary fluid for focusing on a near point, the near point being in closer proximity to the eye than the distant point.

In the second aspect of the invention, preferably the first power is negative, the second power is negative but less negative than the first power, and the third power is positive.

In accordance with the construction of the intraocular lens of these embodiments, multi-focus vision is achieved by the natural motion of the user's eye and/or head. For distant or far vision, the user gazes straight ahead to orient the optical axis substantially parallel to the horizon. In this straight-ahead gaze, the optical axis passes through either the optically transmissive lower liquid or the optically transmissive upper fluid. The refractive index of the fluid through which the optical axis passes and the curvature of the optic body alter the effective power of the lens for focusing.

As the natural inclination to view near objects causes the eye to angle downward for near vision, such as in the case for reading, the upper fluid and the lower liquid move relative to the lens body to pass the optical axis (and visual axis) through both the upper fluid and the lower liquid. The combined refractive indexes of the upper fluid and lower liquid and the curvature of the optic body alter the effective power of the lens for focusing on near objects (at the pp). Thus, as the eye and/or head tilts downward for reading, the position of the eye and the angle of the optical axis of the intraocular lens relative to the horizon changes. This tilting movement alters the power of the lens by intercepting the upper and lower fluids with the optical axis. The effective power of the lens is returned to normal as the optical axis returns to the horizontal orientation and one of the fluids is removed from interception with the optical axis.

In aspects of the invention in which an objective lens is included to provide a telescopic effect, the telescopic optics preferably magnify the image desired to be viewed beyond the borders of the damaged region of the retina (or macula) which is responsible for the scotoma, i.e., in healthy areas of the central retina. As a consequence, although the scotomatous area is not removed from the field of vision, the viewed object preferably is optically altered, e.g., shifted or magnified, so that a greater percentage of the object is viewed peripheral to (i.e., outside of) the scotoma.

According to another aspect of the present invention, a method is provided for reducing the effects of a scotomatous area in a visual field of a person having a retinal degenerative condition. The method comprises inserting an ocular lens into an eye of a person having a retinal degenerative condition characterized by a scotomatous area in a visual field, the ocular lens comprising an optic body, an optically transmissive primary fluid, an optically transmissive secondary fluid, and a fluid interface where the primary and secondary fluids contact one another. The optic body comprises an anterior wall, a posterior wall, and a chamber between the anterior wall and the posterior wall. The optically transmissive primary and secondary fluids are contained in the chamber and have different densities and refractive indexes from one another. Orienting the eye at an intermediate downward gaze passes the visual axis of the eye through the fluid interface to generate a prismatic effect for generating a first image and a second image directed to a first area and a second area of the retina, respectively. At least one of the first and second areas falls at least partially outside, and more preferably completely outside, of a damaged region of the eye responsible for the scotomatous area.

In a preferred embodiment of these and other aspects, the intraocular lens is elastically deformable, such as by folding, to facilitate its insertion into the eye. By elastically, it is meant that the lens has sufficient memory to return to its original shape.

In another preferred embodiment of this invention, the adjustment in effective power of the lens is achieved without any moving parts (other than the flow of the refractive liquids) and without requiring the division of the intraocular lens into separate compartments via internal channels that prevent or inhibit elastic deformation of the lens.

The primary fluid and the secondary fluid used in this method may comprise a first liquid and a second liquid, respectively. In one variation of this aspect, the first density is greater than the second density, and the primary liquid is introduced prior to the secondary fluid. Alternatively, the second density may be greater than the first density, and the secondary liquid may be introduced prior to the primary fluid.

It is to be understood that the aspects for using and making an intraocular lens, as described above, are not the exclusive methods that may be practiced with the intraocular lenses of this invention. Many variations, modifications, and alternative steps and methods to those described above may be used to make and use the intraocular lens of this invention.

### BRIEF DESCRIPTION OF THE DRAWINGS

The accompanying drawings are incorporated in and constitute a part of the specification. The drawings, together with the general description given above and the detailed description of the preferred embodiments and methods given below, serve to explain the principles of the invention. In such drawings:

FIG. 1 is a schematic representation of a human eye with a posterior chamber containing an intraocular lens according to a first embodiment of the invention, in which the eye is gazing straight ahead at the horizon;

FIG. 2 is a schematic representation of the human eye containing the intraocular lens of FIG. 1, in which the eye is angled downward in a reading position;

FIG. 3 is a schematic, enlarged view of the intraocular lens of FIGS. 1 and 2, depicting the lens oriented as shown in FIG. 1;

FIG. 4 is a schematic, enlarged view of the intraocular lens of FIGS. 1 and 2, depicting the lens oriented as shown in FIG. 2;

FIG. 5 is a schematic, enlarged view of an intraocular lens according to a second embodiment of this invention, depicting the lens in the posterior chamber of the eye oriented in a straight-ahead gaze;

FIG. 6 is a schematic, enlarged view of the intraocular lens of the second embodiment of this invention, depicting the lens angled downward in a reading position;

FIG. 7 is a schematic, enlarged view similar to FIG. 3, depicting the intraocular lens in the anterior chamber of the eye;

FIG. 8 is a schematic, enlarged view similar to FIG. 4, depicting the intraocular lens in the anterior chamber of the eye;

FIG. 9 is a schematic, enlarged view similar to FIG. 5, depicting the intraocular lens in the anterior chamber of the eye;

FIG. 10 is a schematic, enlarged view similar to FIG. 6, depicting the intraocular lens in the anterior chamber of the eye;

FIG. 11 is a simplified illustration of an intraocular lens optic body set on a Cartesian coordinate system;

FIGS. 12-14 represent IOL schematics for the examples presented below;

FIGS. 15 and 16 are schematic, enlarged views of an another embodiment of the intraocular lens in straight ahead and downward gazes, respectively;

FIG. 17 is a front view of a modification to the intraocular lens of the first embodiment of the invention;

FIG. 18 is a side sectional view of FIG. 17;

FIG. 19 is a side sectional view of FIG. 17, rotated by 90° upward;

FIG. 20 is a front view of another modification to the intraocular lens of the first embodiment of the invention;

FIG. 21 is a side sectional view of FIG. 20;

FIG. 22 is a side sectional view of FIG. 20, rotated by about 90° upward;

FIG. 23 is a front view of a modification to the intraocular lens of the second embodiment of the invention;

FIG. 24 is a side sectional view of FIG. 23;

FIG. 25 is a side sectional view of FIG. 23, rotated by 90° upward;

FIG. 26 is a front view of another modification to the intraocular lens of the second embodiment of the invention;

FIG. 27 is a side sectional view of FIG. 26;

FIG. 28 is a side sectional view of FIG. 26, rotated by about 90° upward;

FIGS. 29 and 30 are each side sectional views according to additional modified embodiments of the invention;

FIG. 31 shows an example of a Galilean telescopic system; and

FIG. 32 shows an example of a prismatic effect according to an aspect of the invention.

### DETAILED DESCRIPTION OF THE PREFERRED EMBODIMENTS AND PREFERRED METHODS OF THE INVENTION

Reference will now be made in detail to the presently preferred embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

It is to be noted that, as used in the specification and the appended claims, the singular forms “a,” “an,” and “the” include plural referents unless the context clearly dictates otherwise.

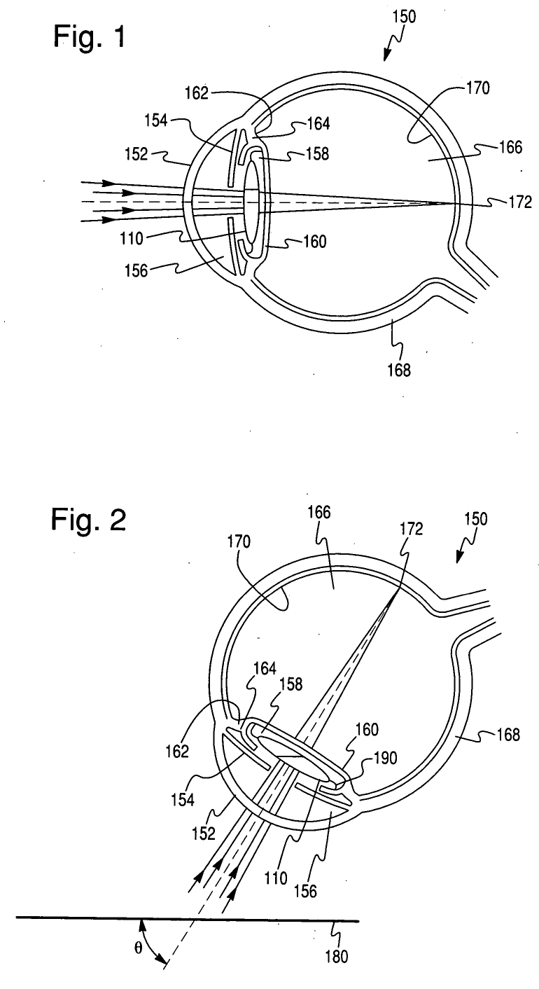

FIGS. 1-4 illustrate an intraocular lens (IOL), generally designated by reference numeral 110, according to a first preferred embodiment of this invention. The intraocular lens comprises an optic body 112 sized and configured to be received in the capsular bag 160 of a human eye 150. The optic body 112 comprises an anterior wall 114, a posterior wall 116, and a chamber 118 between the anterior wall 114 and the posterior wall 116. The chamber 118 is preferably enclosed between the anterior wall 114 and the posterior wall 116, and more preferably is enclosed by a structure consisting of the anterior wall 114 and the posterior wall 116. The anterior and posterior walls 114 and 116 may be, for example, either made as a unitary “integral” piece or may be formed as separate members joined together to form the optic body 112. The optic body 112 has an optical axis 120 intersecting the anterior wall 114 at front vertex (apex) 114a and the posterior wall 116 at rear vertex (apex) 116a. The anterior wall 114 and posterior wall 116 are preferably spherical, although each may be aspheric, and may be produced or modified into an aspheric shape or otherwise to compensate for astigmatism, coma, and higher order aberrations, including double images induced by prism.

In the illustrated embodiment of FIGS. 1-4, the anterior wall 114 is convex and the posterior wall 116 is concave relative to the direction that light travels into the eye 150. (As used herein the terms convexity and concavity refer to the shape of the forward-facing wall surface. Thus, in FIGS. 1-4 the forward-facing surface of the anterior wall 114 has a convex shape and the forward-facing surface of the posterior wall 116 has a concave shape.) However, it is to be understood that in this and other embodiments of the invention, the anterior wall 114 may be concave and/or the posterior wall 116 may be convex, depending upon the desired effective power and refractive properties of the lens 110. Thus, the optic body 112 may take on a convex-concave, convex-convex, concave-convex, or concave-concave configuration, depending upon the particular needs of the individual. Additionally, either the anterior wall 114 or the posterior wall 116 may have a non-curved or flat surface with a radius of curvature equal to zero. In the event one of the walls 114 or 116 is flat, its optical center is assumed to be a region directly opposing the optical center of the other wall.

Because the fluids possess refractive indices, it is possible for one of the walls 114 and 116 to possess no curvature, i.e., to be planar or non-curved. Further, the radii of curvature of the anterior wall 114 and the posterior wall 116 may have the same or different absolute values from each other, depending upon the desired strength of the lens 110. It is also within the scope of the invention to use multiple anterior walls 114 and/or multiple posterior walls 116, and/or to have the anterior wall 114 and/or posterior wall 116 comprised of laminates. Further, the anterior wall 114 and/or posterior wall 116 may be implanted with a lens element or bi-refringent materials. Another possibility is to employ anterior and/or posterior walls with discrete refractive zones, especially concentric zones, such as in the case of Fresnel magnification. However, the optic body 112 of this first embodiment and other embodiments described herein is preferably, although not necessarily, free of interior and exterior channels, especially those that would prevent the deforming or folding of the optic body 112 during surgical insertion.

An optically transmissive upper fluid 122 and an optically transmissive lower liquid 124 are contained in the chamber 118 of the optic body 112. It is preferred in this and other embodiments of the invention that the optically transmissive upper fluid 122 be a liquid, and that the liquids 122 and 124 fill the entire chamber 118, thereby eliminating any gases or free space within the chamber 118. The lower liquid 124 is denser than and has a different refractive index than the upper fluid 122. The upper fluid 122 and the lower liquid 124 are substantially immiscible with each other. As referred to herein, substantially immiscible means that the upper fluid and the lower liquid undergo no or sufficiently small amounts of intermixing that the function of the refractive fluids is performed, i.e., multi-focal sight is obtained by physical tilting of the intraocular lens.

A simplified schematic of the human eye having the intraocular lens 110 of this first embodiment implanted in its posterior chamber 158 of an eye 150 is illustrated in FIGS. 1 and 2. Referring to FIGS. 1 and 2, the eye 150 includes optically transmissive cornea 152, behind which is iris 154. The pupil (unnumbered) is interior to the iris 154 and commonly appears as a black circular area concentrically inward of the iris 154 when viewed from directly in front of the eye 150. The posterior chamber 158 of the eye 150 includes the capsular bag 160, which is shown in this embodiment holding the intraocular lens 110. The chamber between the cornea 152 and the front surface of the capsular bag 160, as shown in FIGS. 1 and 2, is commonly referred to in the art as anterior chamber 156.

Ciliary muscle 162 surrounds the capsular bag 160, and is coupled to the physiological crystalline lens (not shown) by zonules 164. The portion of the posterior chamber 158 behind the capsular bag 160 contains vitreous humor, which is interior to sclera 168. Coating the sclera is the conjunctiva (not shown). Light entering the human eye is converged on the retina 170 at the macula 172, through the optics of the cornea 152 and the intraocular lens 110. As light rays pass through the lens 110, the light rays are bent or refracted to a point at the macula 172 of the retina 170 to provide a clear image. Other light rays that are incident on the retina 170 away from the macula 172 are also detected, usually as part of one's peripheral vision.

The optical axis 120 is situated in the optic body 112 for placement along a light path 121 that enters through and is initially refracted by the cornea 152, then passes through the pupil to the retina 170. An optically transmissive anterior visual zone 114b of the anterior wall 114 defines a surface area through which the light path intersects the anterior wall 114. An optically transmissive posterior visual zone 116b of the posterior wall 116 defines a surface area through which the light path intersects the posterior wall 116. Although the visual zones 114b and 116b may be coextensive with the outer perimeters of the anterior and posterior walls 114 and 116, the visual zones 114b and 116b are more typically smaller in diameter and concentric with the outer perimeters of the anterior and posterior walls 114 and 116. If the lens 110 is positioned in the posterior chamber 156, i.e., posterior to the iris, then incoming light traveling along the light path is refracted by the lens 110 subsequent to passing through the iris 154. Thus, when the lens 110 is in the posterior chamber 158, the iris 154 functions to filter or block a portion of the light that passes through the cornea 152. As referred to herein, the light path through a posterior chamber lens represents the portion of the light that enters through the tear film (not shown) and cornea 152, passes through the pupil and is refracted by the posterior chamber lens 110 to the retina 172. On the other hand, if the lens 110 is positioned in the anterior chamber 156, incoming light traveling along the light path is refracted by the lens 110 before the light passes through the pupil of the iris 154. When the lens is in the anterior chamber 110, the iris 154 serves to filter or block a portion of the light leaving the lens. As referred to herein, the light path through an anterior chamber lens represents the portion of the light that enters through the cornea 152, is refracted by the anterior chamber lens and then passes through the pupil to the retina 172.

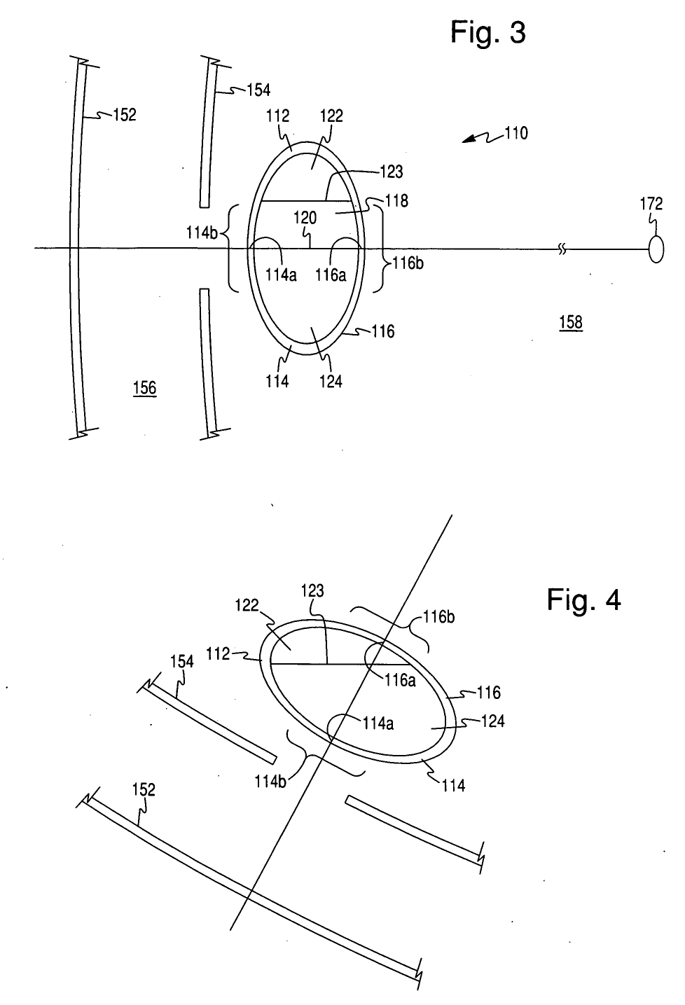

FIGS. 1 and 3 show the intraocular lens 110 of the first embodiment of this invention positioned in the posterior chamber 158 of the eye 150 gazing straight ahead at the pr. In this straight-ahead gaze, the optical axis 120 is parallel to the axis along the horizontal plane 180, or in a horizontal orientation. (Horizontal plane 180 is shown in FIG. 2. As is understood in the art, the eye is usually not rotationally symmetric, so that the optical axis and the visual axis are not co-linear. Hence, if the optical axis is horizontal, the visual axis is usually slightly offset from the horizon. For the purposes of this invention, the straight-ahead gaze refers to the position at which the optical axis is oriented horizontally.) The optically transmissive lower liquid 124 is present in a sufficient amount that orienting the optical axis 120 in the horizontal orientation for distant vision positions the optical axis 120 through the lower liquid 124, and most of the anterior visual zone 114b and the posterior visual zone 116b are immersed in the lower liquid 124. Because the anterior visual zone 114b and posterior visual zone 116b are typically substantially concentric about the front vertex 114a and the rear vertex 116a, contact interface 123 between the lower liquid 124 and the upper fluid 122 is above the vertexes 114a and 116a in the straight-ahead gaze. Preferably, the lower liquid 124 is present in a sufficient amount that in the straight-ahead gaze at least 70 percent, and more preferably all, of the anterior and posterior visual zones 114b and 116b are immersed in the lower liquid 124. Thus, in straight-ahead gaze, light entering the IOL travels along the optical axis and is primarily refracted by denser lower liquid 124. It is believed that any distortion caused by the presence of the fluid interface 123 (or plane of contact of the fluid 122 and liquid 124) in the anterior or posterior visual zone 114b or 116b will be minor and appear as glare to the extent it is even noticeable. The greater the portions of the visual zones 114b and 116b that are immersed in the lower liquid 124 in the straight-ahead gaze, the less the amount of glare or optical aberration, such as coma or halo, if any, that may occur.

The curvatures of the intraocular lens 110 are calculated to account for the refractive index of lower liquid 124 such that light traveling through the eye 150 from the Punctum Remotum may be focused on the macula 172. The anterior or posterior radii of curvature of the lens 110 may be selected depending upon the specific upper fluid 122 and lower liquid 124 chosen and the desired amount of accommodation. It is within the scope of the invention to form a lens which is capable of translating to any desired power for accommodation of eyesight, whether more (+) power or more (−) power upon down gaze. Adjustment of the lens power by modification of the optic body curvature is within the purview of those having ordinary skill in the art.

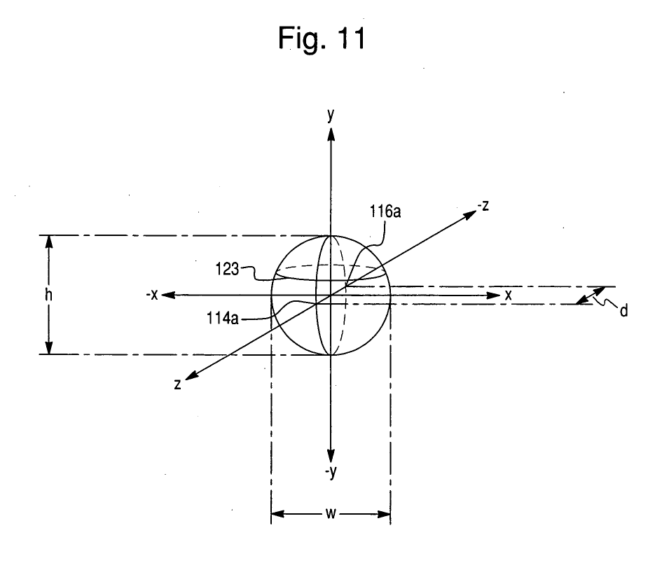

On down gaze, the optical axis 120 rotates to an angle φ relative to the horizontal 180, as shown in FIG. 2. Referring now more particularly to FIG. 11, the lens body 112 is shown in a straight-ahead gaze centered on a Cartesian coordinate system. The lens body 112 has width (w), height (h), and depth (d) on the x, y, and z-axes, respectively. In FIG. 11, the optical axis 120, the front vertex 114a and the rear vertex 116a all rest on the z-axis. Generally, the down gaze involves displacement of the optical axis relative to the horizontal or z-axis by a range of effective angles φ to accomplish the objects of this invention. The effective angles φ may comprise angles throughout a range of 70-90 degrees, more preferably throughout a range of 45-90 degrees, and in some cases as large as angles throughout a range of 30-90 degrees. (Obviously, the natural tilting movement of the human head and/or eye does not pivot its intraocular lenses about a stationary x axis.)

In the down gaze, the optical axis 120 of this first embodiment is positioned at an angle φ relative to horizontal 180 to translate the lower liquid 124 higher on the anterior wall 114 and lower on the posterior wall 116. The upper fluid 122 is present in the chamber 118 in a sufficient amount that, throughout a range of effective angles φ, the upper fluid 122 is translated down the posterior wall 116 so that the optical axis 120 extends through the upper fluid 122 at the back vertex 116a. Preferably, at the range of effective angles, most of the surface area of the anterior visual zone 114b is immersed in the lower liquid 124, and most of the posterior surface area of the posterior visual zone 116b is immersed in the upper fluid 122. More preferably, at the effective angles φ the anterior visual zone 114b has at least 70 percent of its surface area immersed in the lower liquid 124. As used herein, the term “most” may mean “all,” in which case the anterior visual zone 114b has 100 percent of its surface area immersed in the lower liquid 124. (For the purposes of determining the percent immersed surface area, the anterior and posterior visual zones may be assumed to be those for an IOL of this invention implanted into an adult human emmetrope modeled as described in the Optical Society of America Handbook.) Simultaneously, at the effective angles φ the posterior visual zone 116b preferably has at least 70 percent of its surface area, and more preferably all (100 percent) of its surface area, immersed in the upper fluid 122. Under these conditions, the light rays first travels through the lower liquid 124, bathing the anterior visual zone 114b, before traveling through the contact interface 123 then the upper fluid 122 bathing the posterior visual zone 116b, before reaching the retina 170. Because the upper fluid 122 and the lower liquid 124 differ in refractive indices, light traveling through one medium will be refracted more than light traveling through the other medium.

In each of the embodiments described herein, it is preferred that the substantially immiscible fluids/liquids have a sufficiently low viscosity to permit them to freely translate at substantially the same time one's gaze changes from far-to-near and near-to-far. Thus, when the head or eye is returned to straight-ahead gaze, the fluids/liquids translate back to the primary position shown in FIGS. 1 and 3. For the first embodiment, the light rays that focus on the pr pass primarily through the lower liquid 124. This change in power is created without the need for convexity change (e.g., flexing) of the anterior surface 114 or posterior surface 116 of the optic body 112. The change in power is also accomplished without moving the lens 110 relative to the eye 150, i.e., towards or away from the macula 172. Thus, in the first embodiment, on down gaze the upper liquid 122 is translated into the visual axis to provide the desired amount of accommodation for near, e.g., 3 to 9 inches from the eye, and the lens adjusts back to distance focus as straight-ahead gaze is restored.

The range of effective angles φ at which the upper fluid 122 immerses a majority of the surface area of the posterior visual zone 116b is dependent upon the relative amounts of the upper fluid 122 and lower liquid 124 in the chamber 118. For this first embodiment in which the optical axis 120 passes through the lower liquid 124 in the straight ahead gaze (FIGS. 1 and 3), the higher the level of the lower liquid 124 in the chamber 118, the greater the angle φ to contact the upper fluid with the back vertex 116a. Other factors, such as lens thickness, lens radius, and volume shaping, may also affect the effective angle φ.

Referring back to FIG. 11, the width (w), height (h), and depth (d) of the lens body 112 will depend upon several factors, including the sizes of the patient's physiological lens, anterior chamber, and posterior chamber. Generally, the width (w) and height (h) of the lens body 112 may be, for example, in a range of 2.5 mm to 10 mm, more commonly 4.0 mm to 7.5 mm. The width (w) and height (h) are preferably, but not necessarily, the same in dimension. The depth (d) or thickness of the lens body 112 should not be so great as to inhibit implantation into the eye 150. On the other hand, the depth is preferably not so small that the anterior and posterior walls 114 and 116 create significant frictional influence to inhibit fluid translation in the chamber 118 of the lens body 112. The depth (d) may be, for example, at least 0.9 mm.

The anterior visual zone 114b and the posterior visual zone 116b are typically centered concentrically with the front vertex 114a and the rear vertex 116a. Typically, and for the purposes of this invention, the anterior visual zone 114b and the posterior visual zone 116b in an average human eye are about 2 mm to 7 mm in diameter, depending upon the size of the pupil.

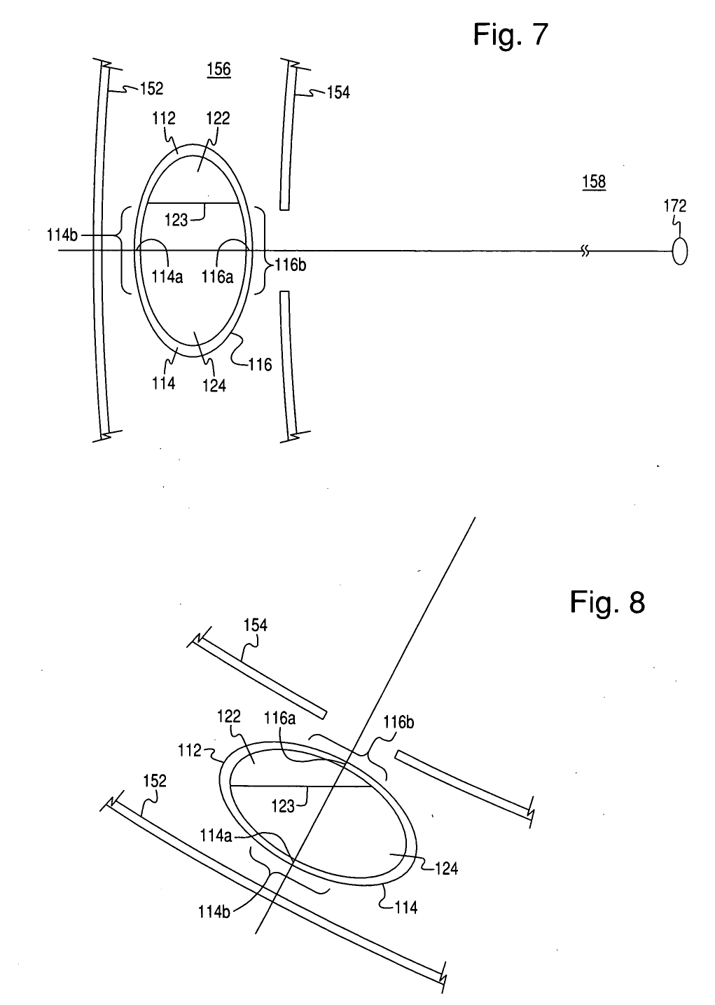

Although the intraocular lens of this first embodiment is illustrated in the posterior chamber 158 of the eye 150, it is to be understood that the lens 110 may be used in the anterior chamber 156, as shown in FIGS. 7 and 8. The intraocular lens 110 in the anterior chamber 156 may be the sole lens in the eye, or may supplement a physiological or synthetic lens placed in the posterior chamber 158. An anterior chamber implantation may be located in front of the iris 154 or between the iris 154 and the front surface of the capsular bag 160. The anterior chamber implantation may be anchored to the iris or in the angle recess.

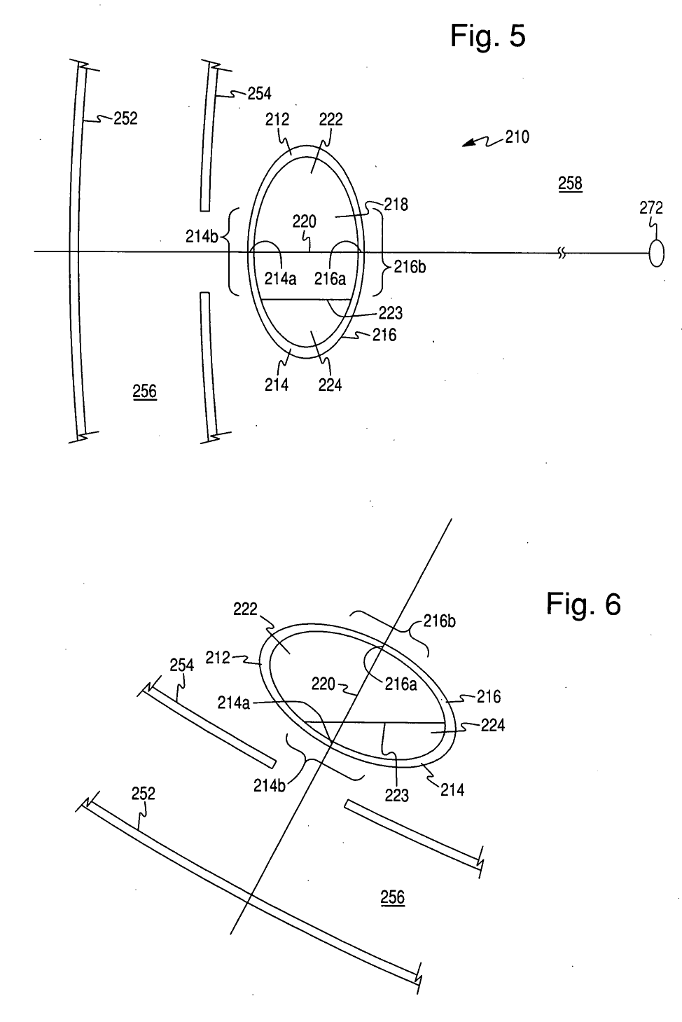

An intraocular lens (IOL) 210 according to a second embodiment of this invention is illustrated in FIGS. 5 and 6. As with the first embodiment, the intraocular lens 210 of the second embodiment comprises an optic body 212 receivable in the capsular bag of a human eye. The optic body 212 comprises an anterior wall 214, a posterior wall 216, and a chamber 218 enclosed between the anterior wall 214 and the posterior wall 216. An optical axis 220 of the optic body 212 intersects the anterior wall 214 at a front vertex 214a and the posterior wall 216 at a rear vertex 216a.

As in the case of the first embodiment, in the second embodiment the intraocular lens 210 is designed for placement in the posterior chamber or anterior chamber of a human eye. The optical axis 220 is situated in the optic body 212 for placement in the human eye along a light path, which passes through the pupil to the retina 270. An optically transmissive anterior visual zone 214b of the anterior wall 214 defines a surface area through which the light path intersects the anterior wall 214. An optically transmissive posterior visual zone 216b of the posterior wall 216 defines a surface area through which the light path intersects the posterior wall 216.

FIG. 5 shows the intraocular lens 210 of the second embodiment of this invention positioned in the posterior chamber 258 of the eye gazing straight ahead at the pr. In this straight-ahead gaze, the optical axis 220 is parallel to the axis along the horizontal plane. The optically transmissive lower liquid 224 is present in a sufficient amount that orienting the optical axis 220 in a horizontal orientation positions the optical axis 220 through the upper fluid 222, and most of the anterior visual zone 214b and the posterior visual zone 216b are immersed in the upper fluid 222. Preferably, the upper fluid 222 is present in a sufficient amount that in the straight-ahead gaze at least 70 percent, and more preferably all, of the anterior and posterior visual zones 214b and 216b are immersed in the upper fluid 222. Thus, in straight-ahead gaze, light entering the IOL travels along the optical axis and is primarily refracted by the upper fluid 222. It is believed that any distortion caused by the presence of the fluid interface (i.e., plane of contact) 223 on the anterior or posterior visual zone 214b or 216b would be minor and appear as glare, to the extent it appears at all. The greater the portions of the visual zones 214b and 216b that are immersed in the upper fluid 222 in the straight-ahead gaze, the less the amount of glare or aberration, if any, that may occur.

The curvatures of the intraocular lens 210 are calculated to account for the refractive index of upper fluid 222 such that light traveling through the eye from the Punctum Remotum may be focused on the macula 272 of the eye. The anterior or posterior radii of curvature of the lens 210 may be selected depending upon the specific upper fluid 222 and lower liquid 224 chosen and the desired amount of accommodation. It is within the scope of the invention to form a lens which is capable of translating to any desired power for accommodation of eyesight, whether more (+) power or more (−) power upon down gaze.

On down gaze, the optical axis 220 rotates to an angle φ relative to the horizontal. As mentioned above, the down gaze generally involves displacement of the optical axis relative to the horizontal or z-axis throughout a range of effective angles φ to accomplish the objects of this invention. The effective angles φ may comprise a range of 70 to 90 degrees, more preferably 45 to 90 degrees, and in some cases over a range comprising 30 to 90 degrees.

In the down gaze, the optical axis 220 of this second embodiment is positioned at an angle φ relative to horizontal to translate the lower liquid 224 higher on the anterior wall 214 and lower on the posterior wall 216. The lower liquid 224 is present in the chamber 218 in a sufficient amount that, at the effective angles φ, the optical axis 220 extends through the lower liquid 224 at the front vertex 214a and the upper fluid 222 at the back vertex 216a. Preferably, in the down gaze most of the surface area of the anterior visual zone 214b is immersed in the lower liquid 224, and most of the surface area of the posterior visual zone 216b is immersed in the upper fluid 222. More preferably, at the effective angles φ (e.g., 70-90 degrees, 45-90 degrees, or 30-90 degrees), the anterior visual zone 214b has at least 70 percent of its surface area, and more preferably 100 percent of its surface area, immersed in the lower liquid 224. Simultaneously, at the effective angles φ the posterior visual zone 216b preferably has at least 70 percent of its surface area, and more preferably 100 percent of its surface area, immersed in the upper fluid 222. Under these conditions, the light rays first travel through the lower liquid 224 bathing the anterior visual zone 214b before traveling through the contact interface 223 and the upper fluid 222 bathing the posterior visual zone 216b, thereafter reaching the retina. Because the upper fluid 222 and the lower liquid 224 differ in refractive indices, light traveling through one medium will be refracted more than light traveling through the other medium.

The range of effective angles φ necessary for displacing the lower fluid 222 to contact the front vertex 214a is dependent upon the relative amounts of the upper fluid 222 and lower liquid 224 in the chamber 218. For this second embodiment in which the optical axis 220 passes through the upper fluid 222 in the straight ahead gaze (FIG. 5), in the illustrated embodiment lower levels of the lower liquid 224 generally will require greater effective angles φ for contacting the lower liquid 224 with the front vertex 214a. Preferably, however, a sufficient amount of the lower liquid 224 is present in this second embodiment that the bi-focal effect is realized throughout at least a range of effective angles of 70-90 degrees.

One particularly advantageous feature embodied in certain aspects of this invention is that orientation of the optical axis perpendicular to the horizon, so that the patient's head is directed straight downward, causes the optical axis to pass through both the upper fluid and the lower liquid, thereby accommodating for near-sight. This feature is especially useful for reading.

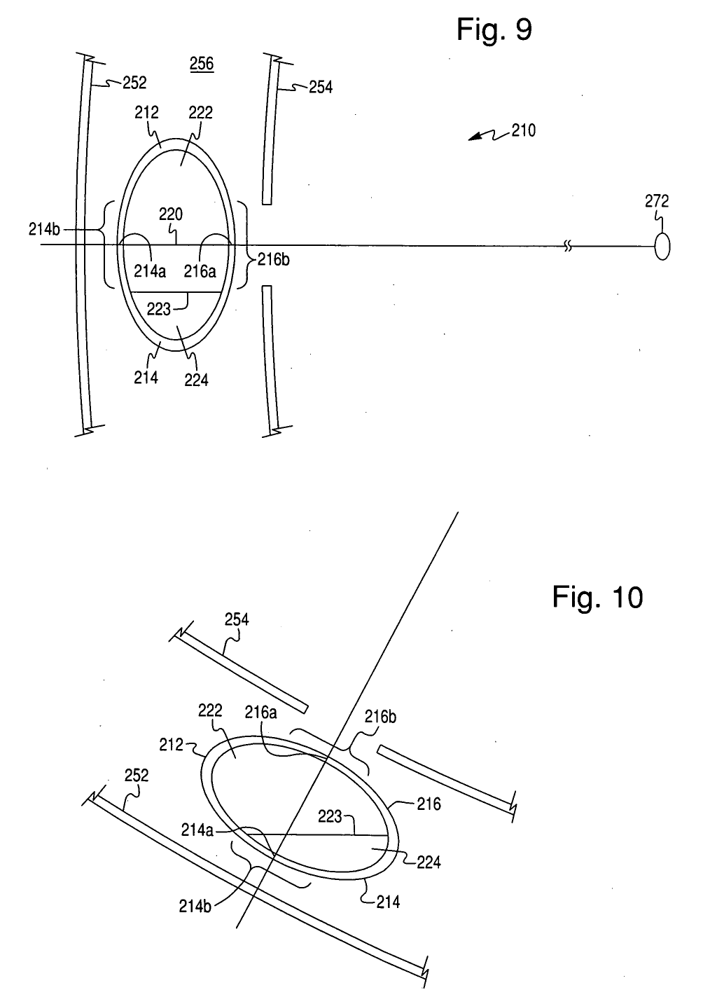

Although the intraocular lens of this second embodiment is illustrated in the posterior chamber 258 of the eye, it is to be understood that the lens 210 may be used in the anterior chamber 256, as shown in FIGS. 9 and 10. The intraocular lens in the anterior chamber may be the sole lens in the eye, or may supplement a physiological or synthetic lens placed in the posterior chamber 258. The intraocular lens may be placed in front of the iris, or between the iris and the capsular bag.

The intraocular lenses of this invention, including those described above and additional embodiments described below, can be used for various eye conditions and diseases, including, for example, aphakia, pseudophakia, anterior cortical cataract extraction (acce), posterior cortical cataract extraction (pcce), accommodative restorative surgery for presbyopes, in refractive correction surgery, and the like. Of particular interest, the embodied intraocular lens is useful for treating retinal degenerative conditions (or “low vision”), and more particularly for reducing the effects of a scotomatous area on a visual field of a person having a retinal degenerative condition.

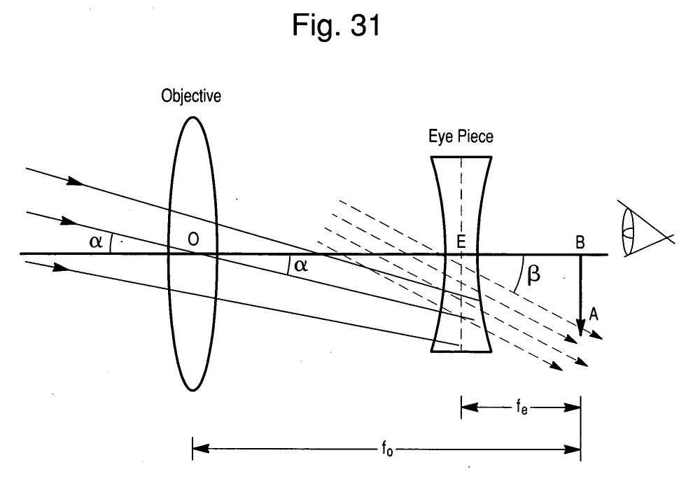

According to embodiments of the invention, treatment of RDCs is accomplished by providing the intraocular lens of the present invention as the ocular lens of a Galilean-type device, and further providing an objective lens in front of the ocular lens for establishing a telescopic benefit and a near-magnifying benefit. The Galilean positive objective lens may comprise, for example, an eyeglass lens, contact lens, and/or an implant in front of the ocular lens. The telescopic benefit is derived from the effective power of the ocular lens in straight-ahead gaze being calculated to be negative in power, and the objective lens in front of the ocular lens being calculated to be positive in power. Preferably, the focal points and/or focal planes of the objective and ocular lenses are coincident with one another, as is the case in a Galilean telescope. The combination of the negative ocular (intraocular) lens and the positive objective lens creates a telescopic power of a Galilean type, provided the focal planes of ocular and objective are coincident, as shown in FIG. 31. As referred to herein and generally understood in the art, a “negative power” lens is a “diverging lens”, i.e., a lens having a cumulative effect of diverging light passing through the lens. On the other hand, a “positive power” lens is a “converging lens”, i.e., a lens having a cumulative effect of converging light rays passing through the lens.

The negative power of the ocular lens is controlled through selection of the fluids and lens curvatures of the ocular lens. By controlling the negative power of the ocular lens and the positive power of the objective lens, a desired magnification can be obtained. In the straight-ahead gaze, the overall telescopic effect of the ocular and objective lens preferably is negative. In the downward gaze, the ocular lens, alone or in combination with the objective lens, provides a near point low vision magnifier. It is preferred that the telescopic effect of the ocular and objective lenses is utilized in the downward gaze. The negative power of the downward gaze, whether attributable to the ocular lens alone or the telescopic effect, preferably is negative, but less negative (i.e., more positive) than the straight ahead gaze for enhancing near vision. The different magnification between straight ahead and down gaze preferably is obtained, for example, by selection of upper and lower fluids having appropriate refractive indices.

The telescopic effect and overall negative power of this preferred embodiment are instrumental in reducing the effects of a scotomatous area of an individual afflicted with a RDC. Without wishing to necessarily be bound by any theory, it is believed that the telescopic optics established by this preferred embodiment magnify the image desired to be viewed beyond the borders of the damaged region of the retina (and more particularly the macula) which is responsible for the scotoma, into healthy areas of the central retina. As a consequence, although the scotomatous area is not removed from the field of vision, the viewed object is shifted, magnified, or otherwise moved so that a greater percentage of the object is viewed outside of the scotoma.

According to a preferred embodiment of the invention, a person with macular degeneration is provided with from about 1.5× to about 5.2× (e.g., about 1.5× to about 5.0×) magnification. More preferably, in straight ahead gaze the magnification is about 1.5× to about 3.0×, and in down gaze the magnification is about 3.0× to about 5.2× (e.g., about 3.0× to about 5.0×). The higher the magnification, the smaller the user's field of view and, therefore, a balance must be reached. Preferably, this balance is dictated by the patient's pupil size, and in particular, maximizing the field of view for the particular pupil size. The greater amount of the pupillary area the ocular lens can fill without introducing an opaque housing into the pupillary line of sight, the better the field of view and the magnified image will appear to the user. Determination of suitable ocular and objective lenses for a particular magnification is within the purview of those skilled in the art. Generally, the focal length of the objective divided by the focal length of the eyepiece equals the magnifying power of the telescope.

In preferred embodiments of the invention, the iris of the natural eye in essence functions as the “housing” of the ocular of the telescope, thereby removing the need for combining the intraocular lens with an artificial housing, such as those used in conventional implanted telescopic devices. Advantageously, omission of an artificial ocular housing can improve the field of vision of a person afflicted with a RDC. For example, the artificial housing of a telescope typically is sized and positioned away from the iris in such a manner as to limit the field of vision, providing a field of vision lesser than that obtainable by the naked eye. Further, the artificial housing of a telescope is not able to account for subtle variations in pupil size due to pupil dilation (e.g., for far vision) and pupil restriction (e.g., for near vision). In embodiments of the present invention in which the iris functions as the telescopic housing of the ocular, the user's field of view is not unduly restricted.

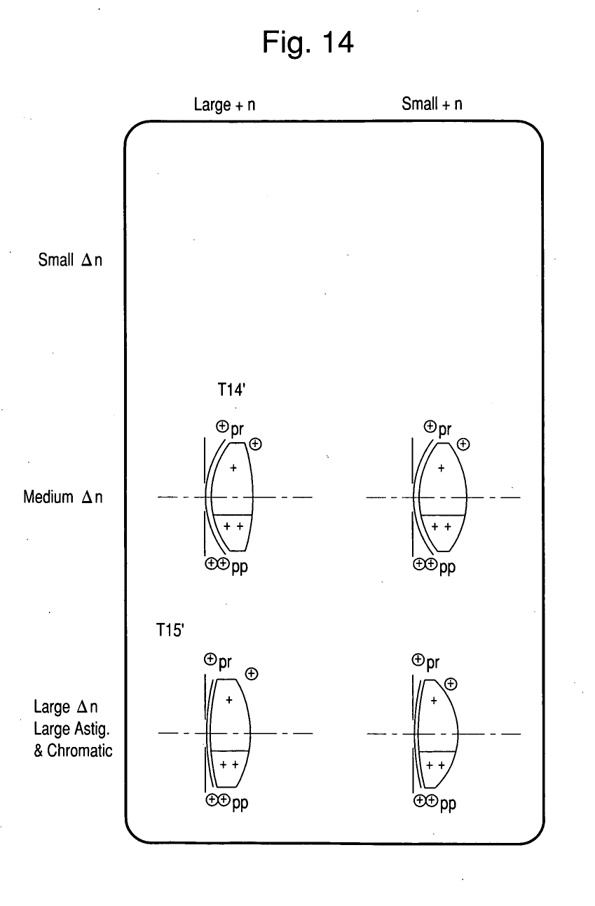

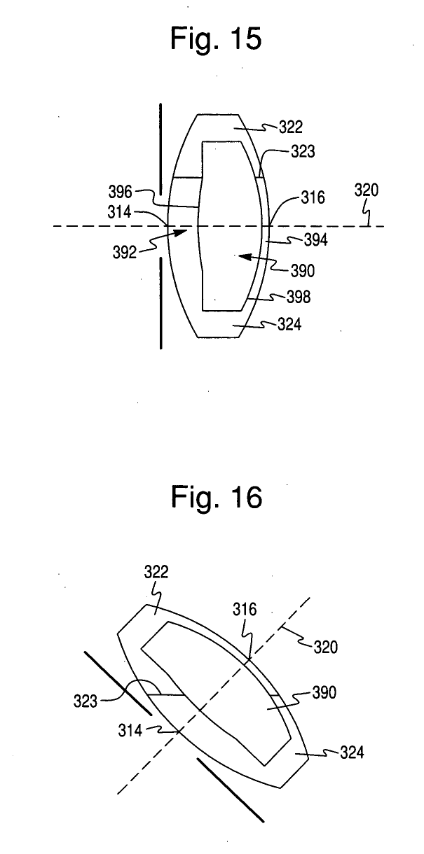

Many modifications and variations to the above-described embodiments are within the scope of the invention. An example of a modification suitable for the first and second embodiments is illustrated in FIGS. 15 and 16. In the interest of brevity and for the purpose of elaborating upon the structure, functions, and benefits of this modification, the description of the first and second embodiments is incorporated herein and not repeated in its entirety. In accordance with this modification, an intraocular lens 310 further comprises at least one supplemental internal lens element 390. The internal lens element 390 may be comprised of, for example, a flexible or rigid material, and may optionally include an internal chamber for holding a liquid or gas. The internal lens element 390 is retained, preferably in a fixed position, inside the intraocular lens body 312. By way of example and not necessarily limitation, webs or filaments may be used for suspending the internal lens element 390 in the fixed position. A first gap 392 is provided between the anterior surface 396 of the internal lens element 390 and the anterior wall 314. A second gap 394 is provided between the posterior surface 398 and the posterior wall 316. Upper fluid 322 and lower liquid 324 are allowed to flow through the gaps 392 and 394.

As shown in FIG. 15, the optically transmissive lower liquid 324 is present in a sufficient amount that orienting the optical axis 320 horizontally positions the optical axis 320 through the lower liquid 324. Most of the anterior visual zone and the posterior visual zone are immersed in the lower liquid 324. The optical axis 320 also passes through the internal lens element 390 in this modified embodiment. The contact interface 323 between the lower liquid 324 and the upper fluid 322 is above the optical axis 320, and preferably above the top edge of the internal lens element 390.

On the down gaze, the optical axis 320 of this modified embodiment is positioned at an angle relative to horizontal to translate the lower liquid 324 higher on the anterior wall 314 and lower on the posterior wall 316. The upper fluid 322 is present in the chamber 318 in a sufficient amount that, throughout a range of effective angles φ, the upper fluid 322 is translated down the posterior wall 316 so that the optical axis 320 extends through the upper fluid 322 at the back vertex 216a. Preferably, at the range of effective angles, most of the surface area of the anterior visual zone is immersed in the lower liquid 324, and most of the posterior surface area of the posterior visual zone is immersed in the upper fluid 322. Under these conditions, the light rays first travel through the lower liquid 324 before traveling through the upper fluid 322. However, in this modified embodiment the optical axis does not pass through the contact interface 323 of the upper fluid 322 and the lower liquid 324. Rather, the light passes through the internal lens element 390, thereby eliminating or substantially eliminating the contact interface 323 from the visual field. As a consequence, to the extent that a meniscus at the contact interface 123 and 223 of the first and second embodiments may contribute to glare or aberration, if any, the internal lens element 390 eliminates or substantially reduces the glare or aberration.

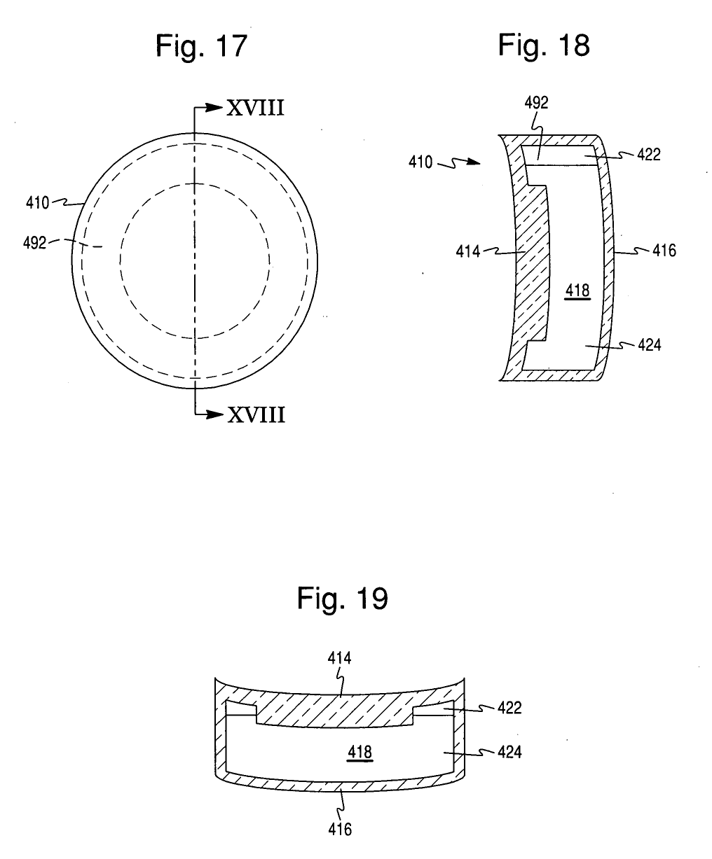

Examples of other modifications suitable for the first and second embodiments and falling within the scope of this invention are illustrated in FIGS. 17-28. In the interest of brevity and for the purpose of elaborating upon the structure, functions, and benefits of these modifications, the descriptions of the first and second embodiments and other modifications described above are incorporated herein and not repeated in their entireties.

In the first embodiment illustrated in FIGS. 1-4, when the eye is tilted upward by a sufficient angle, the upper fluid 122 may enter into the optically transmissive anterior visual zone 114b of the anterior wall 114, causing accommodation from far to near vision. In some instances this effect may be inconsequential or even desirable to the intraocular lens user, depending upon the preferences of the user. However, other intraocular lens users may wish to maintain accommodation for far vision at gazes upward of the horizontal orientation, to as high as φ=−90°, i.e., to vertical.

In accordance with the modification illustrated in FIGS. 17-19, an intraocular lens 410 comprises an anterior wall 414, a posterior wall 416, and a chamber 418 between the anterior wall 414 and the posterior wall 416. The chamber 418 is preferably enclosed between the anterior wall 414 and the posterior wall 416, and more preferably is enclosed by a structure consisting of the anterior wall 414 and the posterior wall 416. The anterior wall 414 and posterior wall 416 are preferably spherical as shown in FIG. 17, although each may be aspheric. The anterior wall 414 includes a dike comprising an annular channel or trench 492, which constitutes part of the chamber 418 and is formed in the anterior wall 414. In the illustrated embodiment, the channel 492 extends 360° around the perimeter of the anterior wall 414. It is to be understood that the channel 492 may extend only a portion of the way around the perimeter of the anterior wall 414, in which case the channel 492 is preferably arcuate. The chamber 418 includes an upper fluid 422 and a lower liquid 424. Preferably, the depth of the upper fluid 422 is smaller than the sectional height of the channel 492, as shown in FIG. 18. As the lens 410 is tilted upward into its vertical position shown in FIG. 19, the upper fluid 422 is maintained in the channel 492, out of the optical centers of the anterior and posterior walls 414 and 424. In this manner, the optical path to the retina passes through the lower liquid 424 while substantially avoiding the upper fluid 422. In downward gaze, the optical path extends through the lower fluid 418 at the anterior wall 414 and the upper fluid 422 at the posterior wall 416.

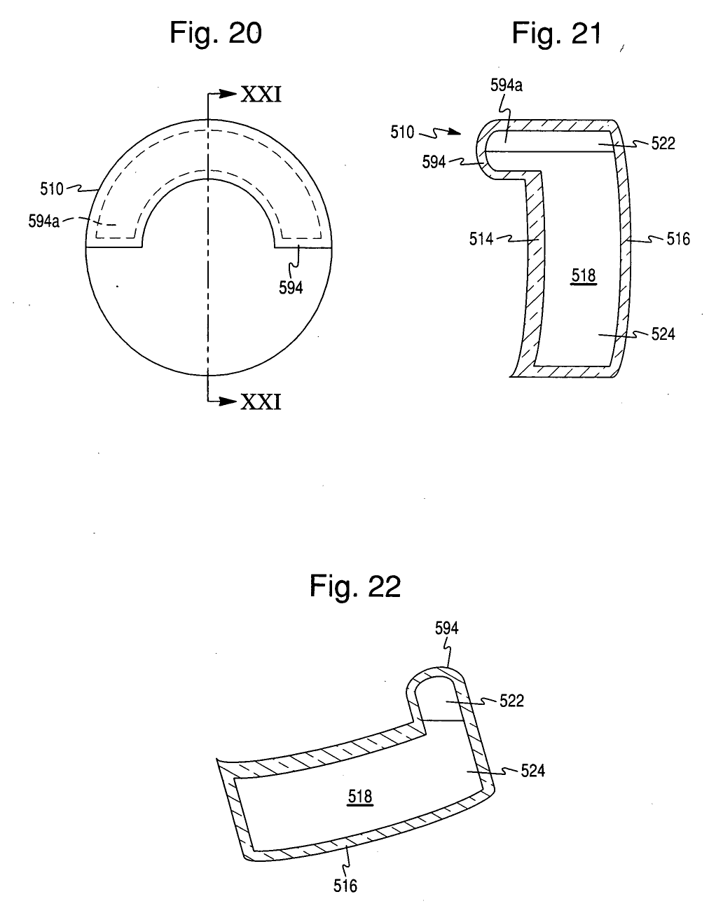

In accordance with the modification illustrated in FIGS. 20-22, an intraocular lens 510 comprises an anterior wall 514, a posterior wall 516, and a chamber 518 between the anterior wall 514 and the posterior wall 516. The chamber 518 is preferably enclosed between the anterior wall 514 and the posterior wall 516, and more preferably is enclosed by a structure consisting of the anterior wall 514 and the posterior wall 516. The anterior wall 514 and posterior wall 516 are preferably spherical as shown in FIG. 20, although each may be aspheric, and may be modified into an aspheric shape or otherwise to compensate for astigmatism, coma, and aberration. Aspheric-shaped lenses may also improve images distorted by a prismatic effect when tilted. The anterior wall 514 includes a dike comprising a protuberance 594, which constitutes part of the anterior wall 514 and has a cavity 594a constituting part of the chamber 518. In the illustrated embodiment, the protuberance 594 is arcuate and extends about 180° around the perimeter of the anterior wall 514. It is to be understood that the protuberance 594 may extend around a small or greater portion or the entirety of the perimeter of the anterior wall 514. The chamber 518 includes an upper fluid 522 and a lower liquid 524. Preferably, the depth of the upper fluid 522 is smaller than the sectional height of the cavity 594a, as shown in FIG. 21. As the lens 510 is tilted upward towards its vertical position shown in FIG. 22, the upper fluid 522 is maintained in the cavity 594a of the protuberance 594, out of the optical zones of the anterior and posterior walls 514 and 524. In this manner, the optical path to the retina passes through the lower liquid 524 while substantially avoiding the upper fluid 522. On the other hand, in downward gaze, the optical path extends through the lower liquid 524 at the anterior wall 514 and the upper fluid 522 at the posterior wall 516.

In the second embodiment illustrated in FIGS. 5 and 6, when the eye is tilted upward by a sufficient angle, the lower liquid 224 may enter into the optically transmissive anterior visual zone 216b of the posterior wall 216, causing accommodation from far to near vision. In some instances this effect may be inconsequential or even desirable to the intraocular lens user, depending upon the preferences of the user. However, some intraocular lens users may wish to maintain accommodation for far vision at gazes upward of the horizontal orientation, to as high as φ=−90°, i.e., to vertical.

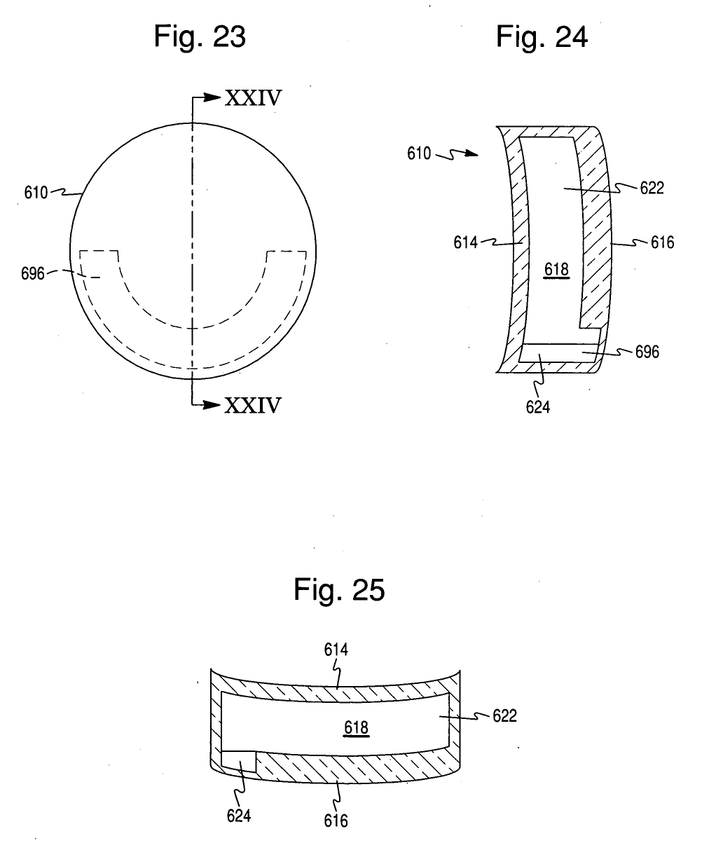

In accordance with the modification illustrated in FIGS. 23-25, an intraocular lens 610 comprises an anterior wall 614, a posterior wall 616, and a chamber 618 between the anterior wall 614 and the posterior wall 616. The chamber 618 is preferably enclosed between the anterior wall 614 and the posterior wall 616, and more preferably is enclosed by a structure consisting of the anterior wall 614 and the posterior wall 616. The anterior wall 614 and posterior wall 616 are preferably spherical as shown in FIG. 23, although each may be aspheric, and may be modified into an aspheric shape or otherwise to compensate for astigmatism. The posterior wall 614 includes a dike comprising a channel or trench 696, which constitutes part of the chamber 618 and is formed in the posterior wall 616. In the illustrated embodiment, the channel 696 is arcuate and extends about 180° about the perimeter of the posterior wall 616. It is to be understood that the channel 696 may extend a greater or less portion or all of the way around the perimeter of the posterior wall 616. The chamber 618 includes an upper fluid 622 and a lower liquid 624. Preferably, the depth of the lower liquid 624 is smaller than the sectional height of the channel 696, as shown in FIG. 24. As the lens 610 is tilted upward into its vertical position shown in FIG. 25, the lower liquid 624 is maintained in the channel 696, out of the visual zones of the anterior and posterior walls 614 and 624. In this manner, the optical path to the retina passes through the upper fluid 622 while substantially avoiding the lower liquid 624. In downward gaze, the optical path extends through the lower liquid 624 at the anterior wall 614 and the upper fluid 622 at the posterior wall 616.

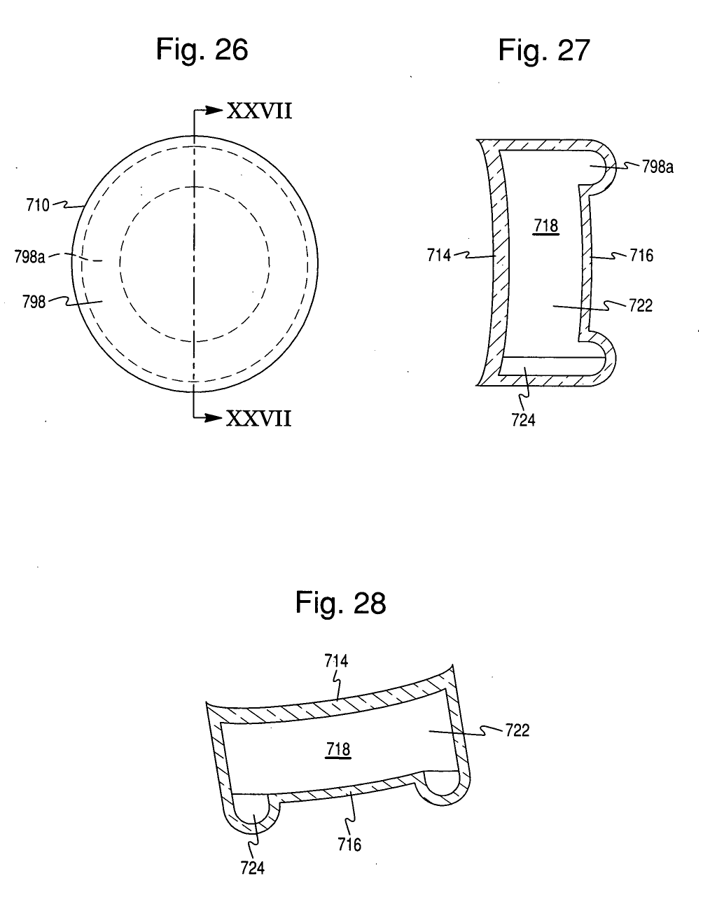

In accordance with the modification illustrated in FIGS. 26-28, an intraocular lens 710 comprises an anterior wall 714, a posterior wall 716, and a chamber 718 between the anterior wall 714 and the posterior wall 716. The chamber 718 is preferably enclosed between the anterior wall 714 and the posterior wall 716, and more preferably is enclosed by a structure consisting of the anterior wall 714 and the posterior wall 716. The anterior wall 714 and posterior wall 716 are preferably spherical as shown in FIG. 26, although each may be aspheric, and may be modified into an aspheric shape or otherwise to compensate for astigmatism. The posterior wall 716 includes a dike comprising an annular protuberance 798, which constitutes part of the posterior wall 716 and has an annular cavity 798a constituting part of the chamber 718. In the illustrated embodiment, the protuberance 798 extends about 360° around the perimeter of the posterior wall 716. It is to be understood that the protuberance 798 may extend a lesser degree around the perimeter of the posterior wall 716, in which case the protuberance is preferably arcuate. The chamber 718 includes an upper fluid 722 and a lower liquid 724. Preferably, the depth of the lower liquid 724 is smaller than the sectional height of the protuberance 798, as shown in FIG. 27. As the lens 710 is tilted upward to its vertical position shown in FIG. 28, the lower liquid 724 is maintained in the protuberance 798, out of the visual zones of the anterior and posterior walls 714 and 724. In this manner, the optical path to the retina passes through the upper fluid 722 while substantially avoiding the lower liquid 724. On the other hand, in downward gaze, the optical path extends through the lower liquid 724 at the anterior wall 714 and the upper fluid 722 at the posterior wall 716.

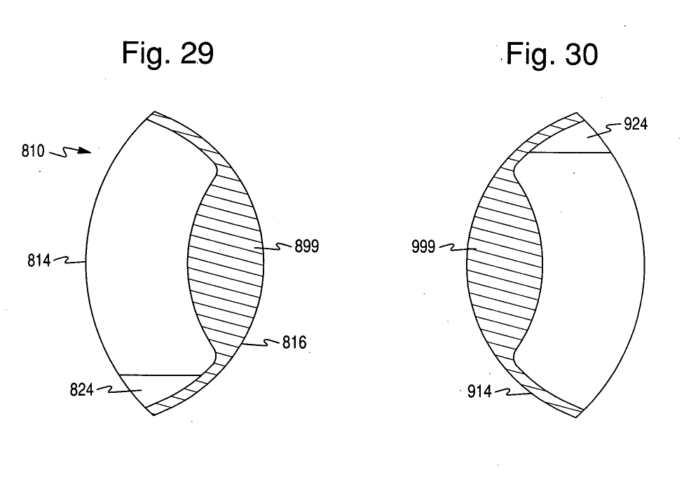

Other designs and configurations may also be practiced for channeling or displacing the secondary fluid away from the optical centers when the optic body is tilted upward relative to the horizontal position. For example and not necessarily limitation, the haptics may be provided with a channel that communicates with the lens chamber. Another example for displacing the secondary fluid away from the optical centers is shown in FIG. 29, in which an intraocular lens having a convex anterior wall 814 and a posterior wall 816 is provided. The posterior wall 816 has a concave outer surface (relative to the direction of light passing into the eye), and has a central bulb portion (or lens) 899 having a generally convex shape. The bulb portion 899 is preferably unitary with the posterior wall 816. As the intraocular lens 810 is tilted downward for reading or near vision, secondary liquid 824 will run forward towards the anterior optical center as described above in connection with the embodiments described above. On the other hand, as the intraocular lens 810 is tilted upward, the bulb portion 899 will direct the secondary liquid 824 towards the bulb edges, away from the posterior optical center. FIG. 30 illustrates a similar embodiment functioning on the same basic principals, except that the bulb portion 999 is part of the anterior wall 914 and the secondary fluid 924 is the upper fluid.

Another embodiment of the invention will now be described with reference to FIGS. 1-4 and 32. The lens of this embodiment comprises an anterior wall and posterior wall (unnumbered) collectively defining a chamber (unnumbered) containing an upper fluid 1122 and a lower liquid 1124 having a fluid interface 1123.

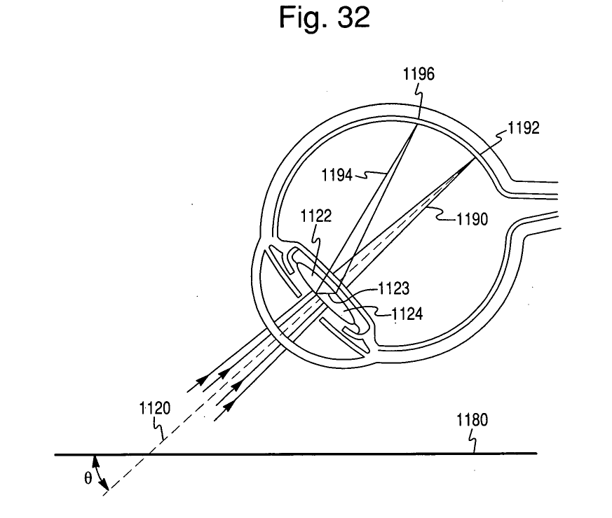

Within the range of effective downward angles φ relative to horizontal plane 1180, the eye passes through an intermediate downward gaze at which the fluid interface 1123 creates a prismatic effect for splitting light rays from a viewed object or objects into a first set of light rays 1190 for producing a first locus of fixation at a first retinal region 1192 and a second set of light rays 1194 for producing the second locus of fixation at a second retinal region 1196. Angles associated with the intermediate downward gaze are typically encountered at the transition from the straight-ahead view to the downward gaze. The first and second retinal regions are preferably different from one another, and more preferably are mutually exclusive, i.e., non-overlapping. At least one and optionally both of the first and second retinal regions 1192, 1196 falls partially or completely outside of a damaged macular region (shown in FIG. 32 as overlapping with 1192) responsible for producing the scotoma. For example, the first retinal region 1192 may comprise an area along the visual axis and thus may overlay the damaged macular region responsible for the scotomatous area, whereas the second retinal region 1196 receiving light rays 1194 as a result of the prismatic effect may lie in a more functional region outside of the damaged macular region 1198 responsible for the scotomatous area.

The range of angles φ associated with the intermediate downward gaze is dependent upon the relative amounts of the upper fluid 1122 and lower liquid 1124 in the chamber. For this first embodiment in which the optical axis 1120 passes through the lower liquid 1124 in the straight ahead gaze (FIGS. 1 and 3), the higher the level of the lower liquid 1124 in the chamber, the greater the angle φ to cause the prismatic effect. Other factors, such as lens thickness, lens radius, and volume shaping may also affect the angle φ. Generally, in preferred embodiments the range of angles φassociated with the prism effect are within (and optionally throughout) a range of 30-60 degrees. The anterior wall 1114 and posterior wall 1116 may be spherical or aspheric. Aspheric lenses may compensate for astigmatism, coma, and higher order aberrations. It should be understood that this aspect of the invention may be practice with other embodiments of the invention comprising a fluid interface in the line of sight, e.g., the embodiments of FIGS. 5-10 and 17-30.

Methods of making optic bodies are well known in the intraocular lens art and are described throughout the literature. These methods, which are suitable for use with the various aspects of the present invention, include, not necessarily by limitation, molding and lathing, with injection molding being perhaps the most commonly employed and well known of these methods. The formation of a molded body with an internal chamber is well known in the injection molding and lathing arts. Methods of gel-capsule manufacture as applied in the pharmaceutical industry may also be applied, as these methods describe introduction of fluids into capsules without leaving vacuum or air space within the capsule. As mentioned above, the anterior and posterior walls may be made as a unitary piece, or separately then joined together, such as by adhesive, fusion, or the like.

The optic body and optional internal lens element 390 preferably comprise a material or materials biologically compatible with the human eye, and capable of injection molding, lathing, or the like. In particular, the materials are preferably non-toxic, non-hemolytic, and non-irritant. The optic body preferably is made of a material that will undergo little or no degradation in optical performance over its period of use. Unlike a contact lens, however, the material does not have to be gas permeable, although it may be. For example, the optic body may be constructed of rigid biocompatible materials, such as, for example, polymethylmethacrylate, or flexible, deformable materials, such as silicones, deformable acrylic polymeric materials, hydrogels and the like which enable the lens body to be rolled, deformed, or folded for insertion through a small incision into the eye. The above list is merely representative, not exhaustive, of the possible materials that may be used in this invention. For example, collagen or collagen-like materials, e.g., collagen polymerized with a monomer or monomers, may be used to form the optic body. However, it is preferred to make the lens body of a material or materials, e.g., elastic, adapted for folding or deformation to facilitate insertion of the intraocular lens into the eye.

The lens surface may be modified with heparin or any other type of surface modification designed to increase biocompatibility and decrease possibility of capsular haze occurring. The lens may also include a “ledge” for reducing formation of capsular haze.

The intraocular lens of this invention may include haptics, which are generally shown in FIGS. 1 and 2, in which the haptics are designated by reference numeral 190. Haptics generally serve to anchor the optics body in place in the eye. Haptics are usually attached directly to the lens body. Various types of haptics are well known in the art, and their incorporation into this invention would be within the purview of an ordinary artisan having reference to this disclosure. Generally, the typical haptic is a flexible strand of nonbiodegradable material fixed to the lens body. By way of example, suitable haptics for this invention may be made of one or more materials known in the art, including polypropylene, poly(methyl methacrylate), and any biocompatible plastic or material in use now or in the future that are used to hold the lens in place. The haptics used with invention may possess any shape or construction adapted or adaptable for use with this invention for securing the lens body in place in the eye. In the posterior chamber, the haptics secure the optical lens within the capsular bag, whereas in the anterior chamber haptics may extend into the area defined between the anterior iris and posterior cornea. For anterior chamber intraocular lenses, it is also within the scope of this invention to use an “iris claw”, which hooks onto the fibers of the iris, or anterior chamber angle fixed haptics.

The upper fluid and the lower liquid may be introduced and retained in the body chamber prior to implanting the IOL into a human eye. The upper and lower fluids may be introduced into the chamber by any technique consistent with the objects of this invention. For example, a syringe or the like may be used for injecting the upper fluid and lower liquid into the chamber. Optionally, an entry port may be provided in the optic body for introducing the upper fluid and lower liquid into the chamber of the optic body. The entry port may be formed during injection molding, by penetrating through one or both of the walls with a suitable hole-making instrument, such as a drill or pin, or it may established by the injecting instrument, e.g., syringe, during introduction of the fluids. The location of the entry port is not critical, i.e., the entry port may be in the anterior wall, posterior wall, or the interface between the walls. Other techniques may also be used to form the optic body.

It is within the scope of the method of this invention to provide the optic body with a vent port for expelling gas (usually air) from inside the optic body chamber as the upper fluid and lower liquid are introduced through the entry port. The vent may be separate from the entry port, or may consist of the entry port such that gas entrapped in the chamber is expelled through the entry port as the upper and lower fluids are introduced into the chamber. Alternatively, the chamber may be evacuated prior to the introduction of the upper fluid and the lower liquid. Subsequent to introducing the upper fluid and lower liquid into the chamber, the entry port and optional vent may be sealed to enclose the chamber in a known manner, such as by fusion or plugging with a compatible material, which may be the same or different than the material from which the optical body is comprised.

It is within the scope of this invention, however, the insert the IOL body into the human eye, then to subsequently inject a portion or all of the upper fluid and the lower liquid into the implanted IOL body in situ. The benefit to this latter variation is that an IOL body that is not filled with fluids/liquids is more amenable to folding and deformation.

Both upper fluid and the lower liquid are preferably optically transmissive, and it is a preferred embodiment that when emulsified by shaking or position change minimal mixing of the upper fluid and the lower liquid occurs, and whatever mixing does occur quickly separates out again. The substantially immiscible upper fluid and lower liquids are preferably optically transparent. It is within the scope of the invention for one or more of the optically transmissive fluids to possess a tint of any color that is not dense enough to significantly impede the transmission of light or the intended objects of this invention. Although the upper fluid is preferably a liquid, it is within the scope of this invention for the upper fluid to be in the form of a gas or vacuum.

This invention is not limited to the use of only two fluids/liquids in the intraocular lens. Three or more fluids of different refractive indexes can be used to create a multi-power, multifocus lens so that objects between far (pr) and near (pp) can be focused upon more clearly. Tri-focals of this invention preferably have three liquids of different densities, with the refractive index of the fluids differing with fluid density.

Fluids that may be used for in the lens body include, but are not limited to, those common to ophthalmic surgery, such as the following: water, aqueous humor, short-chain silicone oils, hyaluron, viscoelastics, polydimethyl siloxane, bis-phenyl propyl dimethicone, phenyl tri-methicone, di-phenyl-di-methyl siloxane copolymer (vinyl-terminated), cyclopentasiloxane, phenyl trimethicone, polydimethyl methyl phenyl siloxane, polymethyl phenyl siloxane, liquid chitosan, heparin, perfluoro-n-octane (perfluoron), perfluoroperhydrophenanthrene, perfluoromethyldecalin, perfluoropentane, perfluoro-1,3-dimethyl cyclohexane, perfluorodecalin, perfluoroperhydro-p-fluorene, and glycerine. It is preferable, but not necessary, that one of the fluids used in the intraocular lens of this invention is water, such as distilled water, to save cost and hazards of broken or ruptured intraocular lenses in vivo.

Many other fluorocarbon liquids may be selected for use as the lower liquid, the upper fluid, or the lower liquid and upper fluid. Representative fluorocarbon fluids that may be used for providing the desired refractive properties of this invention include haloalkanes. Representative haloalkanes that may be useful include trichloromonofluoromethane, dichlorodifluoromethane, monochlorotrifluoromethane, bromotrifluoromethane, dichloromonofluoromethane, monochlorodifluoromethane, dichlorotetrafluoroethane. Other fluorocarbons include 2,2,2-trifluoroethanol, octofluoropentanol-1, dodecafluoroheptanol-1. Other liquids include methanol, acetonitrile, ethyl ether, acetone, ethanol, methyl acetate, propionitrile, 2,2 dimethyl butane, isopropyl ether, 2-methyl pentane, ethyl acetate, acetic acid, D-mannitol, and D-sorbitol.

Many polymethyl/silicon liquid species can be used, including, by way of example, the following: tetrachlorophenylsilsesquixane-dimethyl siloxane copolymer, poly(methylsilsesquioxane, 100% methyl), poly(methylhydridosilsesquioxane, 90%), poly(phenylsilsesquioxane), 100% phenyl, poly(phenyl-methylsilsesquioxane 90% phenyl 10% methyl), dimethicone copolyol PPG-3 oleyl ether (aka alkyl polyether), hydroxymethyl acetomonium PG dimethicone (aka betaine), amino propyl dimethicone (aka amine).

It is within the scope of this invention to select two or more different liquids or fluids as the upper fluid, and to select two or more different liquids as the lower liquid. Dilution of miscible liquids of different indices of refraction may be effective for tailoring the refractive index of the upper fluid or lower liquid phase. Additionally, the dilution of salts, sugars, etc. into the liquids may modify the refractive index. Examples of aqueous salts include sodium chloride, calcium chloride, zinc chloride, potassium chloride, and sodium nitrate (referred to herein as “NaN”). Generally, the concentration of the salts and sugars should be no higher than their saturation points.

These represent chemicals that may be safe within the eye. Other chemicals that are not safe, i.e., biologically compatible with the eye, are less desirable but can have the same visual outcome if maintained within the optical cavity and not exposed to the ocular media within the eye.

As described in connection with the first embodiment above, the intraocular lens can be inserted into the posterior chamber of the human eye, preferably into the capsular bag posterior to the iris to replace the physiological (natural) lens in the capsular bag positioned using known equipment and techniques. Posterior implantation is preferred because, among other reasons, this is the location from which the physiological lens is removed. By way of example, intra-capsular cataract extraction and IOL implantation utilizing clear corneal incision (CCI), phacoemulsification or similar technique may be used to insert the intraocular lens after the physiological crystalline lens has been removed from the capsular bag. The incision into the eye may be made by diamond blade, a metal blade, a light source, such as a laser, or other suitable instrument. The incision may be made at any appropriate position, including along the cornea or sclera. It is possible to make the incision “on axis”, as may be desired in the case of astigmatism. Benefits to making the incision under the upper lid include reduction in the amount of stitching, cosmetic appeal, and reduced recovery time for wound healing. The intraocular lens is preferably rolled or folded prior to insertion into the eye, and may be inserted through a small incision, such as on the order of about 3 mm. It is to be understood that as referred to in the context of this invention, the term “capsular bag” includes a capsular bag having its front surface open, torn, partially removed, or completely removed due to surgical procedure, e.g., for removing the physiological lens, or other reasons. For example, in FIGS. 1 and 2 the capsular bag 160 has an elastic posterior capsule, and an anterior capsular remnant or rim defining an opening through which the physiological lens was removed.

Alternatively, the intraocular lens may be inserted in the anterior chamber between the cornea and the iris. In an anterior chamber implant, the intraocular lens is generally situated forward of, or mounted to, the iris.

When light rays pass between non-opaque media, there is a mathematical description of how light is bent, or refracted. This is termed Snell's Law and is based on the Index of Refraction (IR) of the medium. Different non-opaque media have their own specific index of refraction, and mixed media take on their own individual index of refraction. If two media are placed in contact with one another but do not mix, light will be refracted as it travels from the first medium into the second medium. If a third medium is provided, the light will be refracted again as it passes between the second and third media.

Another aspect of this invention comprises the treatment of one or more residual refractive disorders of the eye after the eye has received an element that allows it to focus, e.g., an intraocular lens, a scleral expansion device, or other element that is designed to substitute for or increase the function of the human accommodative system. In accordance with this aspect, an intraocular lens is implanted into the eye of a human being. Preferably, but not necessarily, the intraocular lens of this aspect of the invention comprises a multi-focal intraocular lens of the one the aspects of the invention described herein. However, it is to be understood that this method may be employed with an accommodative intraocular lenses having conventional or other designs, as well as other intraocular devices that allow the eye to accommodate. The eye is then permitted to heal from the implantation surgery. Sufficient healing to proceed with the altering step will usually take about 3 months, but may be longer or shorter depending upon the recuperative abilities of the eye and the successfulness of the implantation surgery. Residual refractive disorders of the eye are then corrected by altering, e.g., reshaping, a structure of the eye, most preferably the cornea, to improve far distance vision. Treatment of residual refractive disorder after implantation with IOL or other implement that allows for restoration of focus may be achieved by mechanically or chemically altering a structure of the eye, such as the cornea. Representative treatment techniques include, but are not necessarily limited to, light or laser refractive surgery of the cornea (including PRK [photo-refractive keratectomy], LASIK [laser intra-stromal keratectomy], LASEK [laser epithelial keratectomy]) performed with excimer lasers, YAG (yttrium-aluminum-garnet) lasers or other ablative lasers of single frequency or frequency modulation including but not limited to frequency doubling or tripling, thermal keratoplasty, conductive keratoplasty including radio waves, corneal ring segments. Each of these techniques on a human eye having a natural lens is well known and the art and described in various literature documents too numerous to list. One example of a literature document describing techniques for shaping the cornea is U.S. Pat. No. 4,994,058. Application of these techniques on a human eye containing a corrective element, such as an intraocular lens, may be performed by those of ordinary skill in the refractive correction arts without undue experimentation.

The lenses of the various aspects and embodiments described herein may be used in one or both eyes of the subject. For example, it may be desirable to have the lens serve as the ocular of a telescope by being made negative and placed in one eye, but not the other.

### EXAMPLES

All examples were modeled on the Zemax Version 10.0 optical design program, SE edition, from Focus Software, Inc.

The human eye was first modeled as a typical or schematic adult human emmetrope, as described in the Optical Society of America Handbook. Each of the models described below is for a posterior chamber IOL design. The following assumptions were made for the human eye for the purposes of the calculations. The model was assumed to have spherical surfaces only (whereas the real cornea and lens are actually aspherics). Each structure of the schematic human eye was assumed to be made of a material having a uniform or homogenous index (whereas in the real human eye, the index of refraction may vary somewhat through each structure of the eye). The model also assumed that the capsular bag walls were very thin and parallel, i.e., non-existent. The lens was assumed to have symmetric radius, i.e., spherical. The pr was assumed to be 10 meters. Three wavelengths with equal weighting were used for optimization and evaluation: 510 nm, 560 nm, and 610 nm to provide a simple approximation of the human photopic response. Walker, Bruce H., Optical Design for visual Systems, SPIE Press (2000). The Abbe wavelength dispersion is assumed to be 55.0 for all natural materials. The indices at other wavelengths were calculated based on n_{D} and the dispersion value. Modeling was performed for small pupil sizes of 1.5 mm. The initial values assumed for the eye are listed below in Table 1.

TABLE 1

| | Radius | Thickness | Refr. Index | |
|---|---|---|---|---|
| Surface | (mm) | (mm) | (@589 nm) | Material |
| Anterior | 7.80 | 0.55 | 1.3771 | Cornea |
| Cornea | | | | |
| Posterior | 6.50 | 3.05 | 1.3374 | Aqueous |
| Cornea | | | | Humor |
| Anterior Lens | 10.20 | 4.00 | 1.4200 | Natural lens |
| | 20.83* | | | |
| Posterior Lens | −6.00 | 16.6 | 1.3360 | Vitreous |
| | −4.26* | 16.80* | | Humor |
| Retina | −12.67* | | | |

*italics indicates values optimized through Zemax program, under assumed conditions as listed.

The above assumptions and conditions were maintained for the IOL designs, with the natural lens replaced by the IOL. The overall length of the eye models was kept constant. The IOL thickness was allowed to adjust during optimization, but not to exceed 4.0 mm.

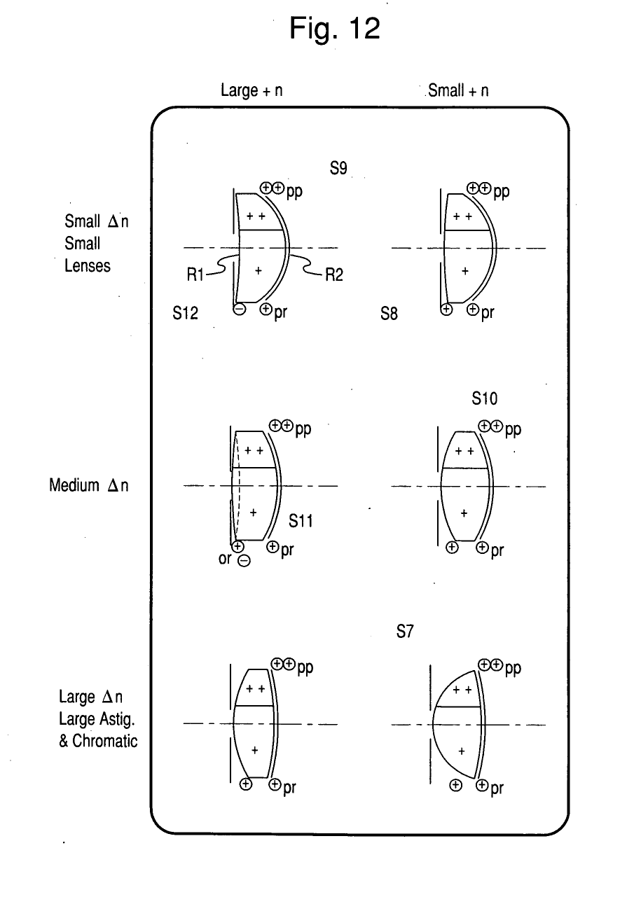

According to one set of preferred IOL designs illustrated in FIG. 12, the lower liquid is the primary liquid and has a lesser refractive index than the upper liquid. Accordingly, in this preferred embodiment the upper liquid has a greater refractive index and imparts accommodative power (+power) on down gaze by increasing the effective power of the posterior IOL surface. Models were made for the combinations of fluids in Table 2. The index of refraction value were either taken as reported in the literature at 37° C. (body temperature) in a saturated solution, or were estimated based on calculations using three (3) wavelengths (of 510 nm, 560 nm, and 610 nm).

TABLE 2

| | | Upper | | | | | |
|---|---|---|---|---|---|---|---|
| Label | Lower Liquid | Liquid | n_{D}1** | n_{D}2** | R1*** | R2*** | Thickness**** |
| S9 | Aq-NaN | PDMS- | 1.38543 | 1.39908 | −43.750 | −2.52 | 2.12 |
| | | (37° C.) | | | | | |
| S8 | Aq-NaCl | PDMS | 1.37794 | 1.39908 | 6.081 | −3.65 | 2.32 |
| | | (37° C.) | | | | | |
| S12 | Aq-CaCl | Mineral | 1.44287 | 1.46408 | −14.770 | −3.98 | 1.62 |
| | | Oil | | | | | |
| S10 | Aq-KCl | PDMS- | 1.36035 | 1.39908 | 1.875 | −6.82 | 1.58 |
| | | (37° C.) | | | | | |
| S11 | Aq-ZnCl | Mineral | 1.40229 | 1.46408 | 5.837 | −9.00 | 3.54 |
| | | Oil | | | | | |
| S7 | Aq-NaCl | Mineral | 1.37789 | 1.46408 | 3.029 | −14.00 | 2.30 |
| | | Oil | | | | | |

**n_{D}1 and n_{D}2 are refractive index of lower liquid and the upper liquid, respectively, at or about its saturation limit at 589 nm wavelength.
***R1 and R2 are the radius of curvature of the anterior surface and the posterior surface, respectively, in millimeters.
****Lens thickness was measured in millimeters.

The shapes of the anterior and posterior walls were calculated for hypothetical cases by modifying the adult human emmetrope model to simulate an IOL. The crystalline lens material was replaced with the lower fluid to simulate horizontal pr gaze (at 10 m), and the pp (250 mm) was modeled in a directly vertical 90° downward gaze angle using two liquids with the interface perpendicular to the optical axis. The posterior radius of the lens was selected to obtain the needed change of power with the upper liquid introduced to accommodate for pp (at about 250 mm). Other assumptions listed above for the model eye were also made. Gaze angles of less than 90° were then evaluated without re-optimizing the model parameters. Specifically, gaze angles of 50° and 70° were investigated. The 90°, 70°, and 50° gaze angles were each evaluated at the following five field points of 0°, ±7.5°, and ±15°. The root mean square (RMS) of each spot radius value was then recorded. Reported below are the averages of the five field values, and the RMS for the on-axis (0°) field point. All RMS values are in microns.

TABLE 3

| | RMS Spot: | | | RMS Spot: | | |
|---|---|---|---|---|---|---|
| | Average of 5 Fields | | | On-Axis Value | | |
| Label | 90° | 70° | 50° | 90° | 70° | 50° |
| S9 | 4.81 | 5.14 | 7.26 | 3.87 | 4.47 | 6.97 |
| S8 | 4.78 | 4.89 | 7.93 | 3.21 | 4.00 | 8.16 |
| S12 | 4.03 | 4.03 | 5.94 | 2.88 | 3.11 | 5.31 |
| S10 | 9.28 | 9.45 | 15.59 | 5.16 | 6.84 | 15.71 |
| S11 | 5.41 | 6.164 | 17.95 | 3.45 | 5.86 | 18.99 |
| S7 | 7.29 | 8.79 | 26.29 | 4.53 | 8.37 | 27.67 |

Smaller RMS values generally indicate less aberration and better focus on the retina. Generally, values less than 7.00 microns are preferred for the assumed conditions.

The IOL schematics are laid out as though plotted on a chart, with the actual fluid's refractive index along the horizontal axis (abscissa) and the difference in the index values of the two fluids on the vertical axis (ordinate). Internal to the lens schematics, the fluids are labeled with the following symbols:
+ a liquid having an index of refraction greater than the humors in which the IOL is immersed when implanted;
++ a liquid having an index of refraction greater than the humors and the adjacent “+” liquid;
− a liquid having an index of refraction lower than the humors;
−− a liquid having an index of refraction lower than the humors and the adjacent “−” liquid.

The cornea (not shown) is to the left of the IOL schematics, and the iris is shown immediately to the left of the IOL schematics. The surface that produces the optical power change (pr to pp adaptation) is shown with a double line.

As shown in FIG. 12, the IOL schematics for this embodiment preferably had concave/concave, convex/concave walls, or flat/concave walls. Fluid combinations S9 and S10 were less preferred due to the steep curvatures of R1 (anterior surface) or R2 (posterior surface).

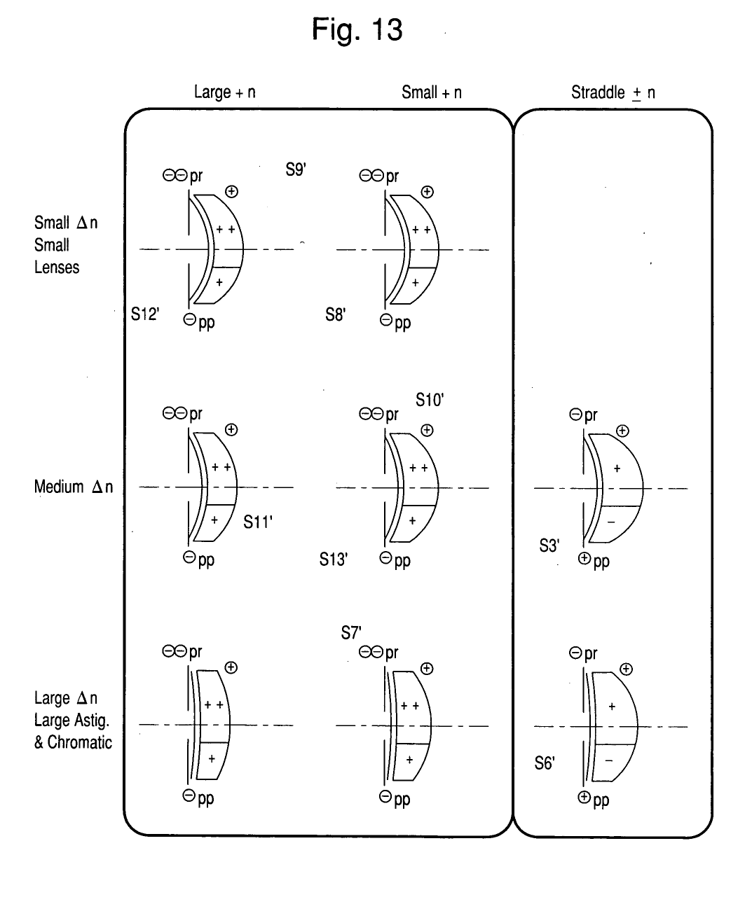

According to another set of preferred IOL designs illustrated in FIG. 13, the upper liquid is the primary liquid and has a greater refractive index than the lower liquid. Hence, the lower liquid imparts accommodative power (+power) on down gaze by increasing the effective power of the lens. Models were made for the following combinations of fluids:

TABLE 4

| | Lower | Upper | | | | |
|---|---|---|---|---|---|---|
| Label | Liquid | Liquid | n_{D}1 | n_{D}2 | R1 | R2 |
| S9′ | PDMS- | Aq-NaN | 1.39908 | 1.38543 | −2.90 | −1.703 |
| | (37° C.) | | | | | |
| S8′ | PDMS | Aq-NaCl | 1.39908 | 1.37794 | −4.40 | −2.032 |
| | (37° C.) | | | | | |
| S12′ | Mineral | Aq-CaCl | 1.46408 | 1.44287 | −4.45 | −2.770 |
| | Oil | | | | | |
| S10′ | PDMS- | Aq-KCl | 1.39908 | 1.36035 | −8.10 | −2.458 |
| | (37° C.) | | | | | |
| S11′ | Mineral | Aq-ZnCl | 1.46408 | 1.40229 | −12.95 | −4.296 |
| | Oil | | | | | |
| S13′ | Mineral | Aq-NaN | 1.46408 | 1.38543 | −16.50 | −4.564 |
| | Oil | | | | | |
| S7′ | Mineral | Aq-NaCl | 1.46408 | 1.37789 | −18.17 | −4.661 |
| | Oil | | | | | |
| S5′ | PDMS | Water | 1.39908 | 1.33100 | −14.35 | −2.760 |
| | (37° C.) | (37° C.) | | | | |
| S6′ | Mineral | Water | 1.46408 | 1.33100 | −28.40 | −5.032 |
| | oil | (37° C.) | | | | |

The shapes of the anterior and posterior walls were calculated for hypothetical cases by modifying the adult human emmetrope model to simulate an IOL. The crystalline lens material was replaced with the upper fluid to simulate horizontal pr gaze (at 10 m), and the pp (at about 250 mm) was modeled in a directly vertical 90° downward gaze angle using two fluids with the interface perpendicular to the optical axis. The anterior radius of the lens was selected to obtain the needed change of power with the lower liquid introduced to accommodate for pp. Again, assumptions made above for the model eye were applied, as needed. Gaze angles of less than 90° were then evaluated without re-optimizing the model parameters.

TABLE 5

| | RMS Spot: | | | RMS Spot: | | |
|---|---|---|---|---|---|---|
| | Average of 5 Fields | | | On-Axis Value | | |
| Label | 90° | 70° | 50° | 90° | 70° | 50° |
| S8′ | 7.06 | 7.17 | 8.61 | 6.23 | 6.38 | 7.77 |
| S12′ | 5.88 | 5.91 | 6.55 | 4.56 | 4.69 | 5.55 |
| S10′ | 5.24 | 5.54 | 10.67 | 4.23 | 4.82 | 10.20 |
| S11′ | 4.03 | 4.73 | 13.33 | 2.73 | 3.92 | 12.78 |
| S13′ | 3.94 | 5.18 | 17.23 | 2.58 | 4.40 | 16.47 |
| S7′ | 3.97 | 5.59 | 13.60 | 2.63 | 4.87 | 18.25 |
| S5′ | 4.66 | 5.80 | 17.64 | 3.54 | 5.26 | 17.10 |
| S6′ | 4.11 | 8.39 | 31.63 | 2.68 | 7.74 | 30.06 |

As shown in FIG. 13, the IOL schematics for these examples preferably had concave/concave walls, with the anterior surface concavity more pronounced than in FIG. 12. Fluid combinations S5′, S8′, S9′, S10′, and S12′ were less preferred due to the small sizes of the IOL R1 and/or R2.

According to another set of preferred IOL designs illustrated in FIG. 14, the upper liquid is the primary liquid and has a smaller refractive index than the lower liquid. Models were made for the combinations of fluids set forth in Table 6, with the corresponding results reported in Table 7:

TABLE 6

| | Lower | Upper | | | | |
|---|---|---|---|---|---|---|
| Label | Liquid | Liquid | nD1 | nD2 | R1 | R2 |
| T14′ | PDMS- | Aq-CaCl | 1.39908 | 1.44287 | 9.19 | −4.750 |
| | (37° C.) | | | | | |
| T15′ | PDMS | Glycerol | 1.39908 | 1.47238 | 15.30 | −4.022 |
| | (37° C.) | | | | | |

TABLE 7

| | RMS Spot: | | | RMS Spot: | | |
|---|---|---|---|---|---|---|
| | Average of 5 Fields | | | On-Axis Value | | |
| Label | 90° | 70° | 50° | 90° | 70° | 50° |
| T14′ | 5.14 | 7.31 | 19.56 | 3.34 | 4.43 | 14.81 |
| T15′ | 4.65 | 8.29 | 28.38 | 3.04 | 5.24 | 23.17 |

Convex/concave wall structures were preferred for these examples.

It was observed from modeling that the tilt of the fluid interface (downward gazes not equal to 90°) may cause astigmatism and chromatic aberrations, which can be minimized by decreasing the differential value between the fluid indices. However, too small an index differential may require compensation vis-à-vis reduction to the radii of curvature. Reduction in radii of curvature may produce IOLS have diameters that are too small and increased spherical aberration and coma. Thus, a fundamental tradeoff exists between the normal aberrations (no tilt of the fluids) and the performance as the gaze departs from directly downward.

The lens schematics illustrated in the accompanying drawings are intended to show general trends, and are not intended or shown as precise designs. The illustrated schematics are also not intended to be exhaustive of the scope of possible IOL body designs within the scope of this invention.

The foregoing detailed description of the preferred embodiments of the invention has been provided for the purposes of illustration and description, and is not intended to be exhaustive or to limit the invention to the precise embodiments disclosed. The embodiments were chosen and described in order to best explain the principles of the invention and its practical application, thereby enabling others skilled in the art to understand the invention for various embodiments and with various modifications as are suited to the particular use contemplated. It is intended that the scope of the invention cover various modifications and equivalents included within the spirit and scope of the appended claims.

1. A method for optically altering an image peripheral to a scotomatous area in a visual field of a person having a retinal degenerative condition, comprising:
inserting an ocular lens into an eye of a person having a retinal degenerative condition characterized by a scotomatous area in a visual field, the ocular lens comprising an optic body, an optically transmissive primary fluid, and an optically transmissive secondary fluid, the optic body comprising an anterior wall, a posterior wall, and a chamber between the anterior wall and the posterior wall, the optically transmissive primary and secondary fluids contained in the chamber and having different densities and refractive indexes from one another.

2. A method according to claim 1, wherein the ocular lens magnifies an image of a viewed object so that a greater percentage of the object is viewed outside of the scotomatous area.

3. A method for optically altering an image peripheral to a scotomatous area in a visual field of a person having a retinal degenerative condition, comprising:
inserting an ocular lens into an eye of a person having a retinal degenerative condition characterized by a scotomatous area in a visual field, the ocular lens comprising an optic body, an optically transmissive primary fluid, and an optically transmissive secondary fluid, the optic body comprising an anterior wall, a posterior wall, and a chamber between the anterior wall and the posterior wall, the optically transmissive primary and secondary fluids contained in the chamber and having different densities and refractive indexes from one another, the ocular lens having a first power in straight ahead gaze and a second power in down gaze; and
providing an objective lens having a third power in front of the ocular lens to establish a telescopic effect;
wherein orienting the eye in a generally straight ahead gaze for far vision passes the visual axis of the eye through the primary liquid, but not the secondary liquid, for focusing on a distant point; and
wherein moving the eye into a downward gaze passes the visual axis through the primary fluid and the secondary fluid for focusing on a near point, the near point being in closer proximity to the eye than the distant point.

4. A method according to claim 3, wherein the ocular lens magnifies an image of a viewed object so that a greater percentage of the object is viewed outside of the scotomatous area.

5. A method according to claim 4, wherein in the generally straight ahead gaze the ocular and objective lenses collectively provide a magnification of about 1.5× to about 3.0×, and in downward gaze the magnification is about 3.0× to about 5.2×.

6. A method according to claim 4, wherein the primary fluid and the secondary fluid comprise a first liquid and a second liquid, respectively.

7. A method according to claim 6, wherein a contact interface is interposed between the first liquid and the second liquid, and wherein orienting the optical axis for near vision throughout a range of effective downward angles relative to a horizontal orientation positions the optical axis to extend through the contact interface.

8. A method according to claim 6, wherein the first density is greater than the second density, and wherein orienting the optical axis throughout the range of effective downward angles positions the optical axis to extend through the primary fluid at the anterior optical center and the secondary fluid at the posterior optical center.

9. A method according to claim 6, wherein the second density is greater than the first density, and wherein orienting the optical axis throughout the range of effective downward angles positions the optical axis to extend through the secondary fluid at the anterior optical center and the primary fluid at the posterior optical center.

10. A method according to claim 6, wherein the secondary fluid is contained in the chamber of the optic body in a sufficient amount that orienting the optical axis for near vision throughout a range of at least 70 degrees to 90 degrees relative to the horizon orientation positions the optical axis to extend through the primary fluid and the secondary fluid.

11. A method according to claim 6, wherein the secondary fluid is contained in the chamber of the optic body in a sufficient amount that orienting the optical axis for near vision throughout a range of at least 45 degrees to 90 degrees relative to the horizon orientation positions the optical axis to extend through the primary fluid and the secondary fluid.

12. A method according to claim 6, wherein the secondary fluid is contained in the chamber of the optic body in a sufficient amount that orienting the optical axis for near vision throughout a range of at least 30 degrees to 90 degrees relative to the horizon orientation positions the optical axis to extend through the primary fluid and the secondary fluid.

13. A method according to claim 6, wherein the range of effective downward angles encompasses 90 degrees from the horizontal orientation.

14. A method according to claim 3, wherein the first power is negative, the second power is negative but less negative than the first power, and the third power is positive.

15. A method according to claim 14, wherein the primary fluid and the secondary fluid comprise a first liquid and a second liquid, respectively.

16. A method according to claim 15, wherein a contact interface is interposed between the first liquid and the second liquid, and wherein orienting the optical axis for near vision throughout a range of effective downward angles relative to a horizontal orientation positions the optical axis to extend through the contact interface.

17. A method according to claim 15, wherein the first density is greater than the second density, and wherein orienting the optical axis throughout the range of effective downward angles positions the optical axis to extend through the primary fluid at the anterior optical center and the secondary fluid at the posterior optical center.

18. A method according to claim 15, wherein the second density is greater than the first density, and wherein orienting the optical axis at the range of effective downward angles positions the optical axis to extend through the secondary fluid at the anterior optical center and the primary fluid at the posterior optical center.

19. A method according to claim 15, wherein the secondary fluid is contained in the chamber of the optic body in a sufficient amount that orienting the optical axis for near vision throughout a range of at least 70 degrees to 90 degrees relative to the horizon orientation positions the optical axis to extend through the primary fluid and the secondary fluid.

20. A method according to claim 15, wherein the secondary fluid is contained in the chamber of the optic body in a sufficient amount that orienting the optical axis for near vision throughout a range of at least 45 degrees to 90 degrees relative to the horizon orientation positions the optical axis to extend through the primary fluid and the secondary fluid.

21. A method according to claim 15, wherein the secondary fluid is contained in the chamber of the optic body in a sufficient amount that orienting the optical axis for near vision throughout a range of at least 30 degrees to 90 degrees relative to the horizon orientation positions the optical axis to extend through the primary fluid and the secondary fluid.

22. A method according to claim 15, wherein the range of effective downward angles encompasses 90 degrees from the horizontal orientation.

23. A method for reducing the effects of a scotomatous area in a visual field of a person having a retinal degenerative condition, comprising:
inserting an ocular lens into an eye of a person having a retinal degenerative condition characterized by a scotomatous area in a visual field, the ocular lens comprising an optic body, an optically transmissive primary fluid, an optically transmissive secondary fluid, and a fluid interface where the primary and secondary fluids contact one another, the optic body comprising an anterior wall, a posterior wall, and a chamber between the anterior wall and the posterior wall, the optically transmissive primary and secondary fluids contained in the chamber and having different densities and refractive indexes from one another; and
orienting the eye at an intermediate downward gaze to pass the visual axis through the fluid interface to generate a prismatic effect for generating a first image and a second image directed to a first area and a second area of the retina, respectively, at least one of the first and second areas falling at least partially outside of a damaged region of the eye responsible for the scotomatous area.

24. A method according to claim 23, wherein the primary and secondary fluids are contained in the chamber of the optic body in effective amounts to generate the prismatic effect within a range of 30 degrees to 60 degrees relative to a horizontal orientation.

25. A method according to claim 23, further comprising providing an objective lens in front of the ocular lens, wherein the ocular lens has a negative power and the objective lens has a positive power, and wherein the ocular and objective lenses collectively provide a telescopic effect.

26. A method according to claim 23, wherein:
orienting the eye in a generally straight ahead gaze passes the visual axis through the primary liquid, but not the secondary liquid; and
moving the eye into a downward gaze passes the visual axis through the primary liquid and the secondary liquid.

27. A method according to claim 26, wherein the primary fluid and the secondary fluid comprise a first liquid and a second liquid, respectively.

28. A method according to claim 27, wherein the first density is greater than the second density, and wherein orienting the eye at the intermediate downward gaze positions the optical axis to extend through the primary fluid at the anterior optical center and the secondary fluid at the posterior optical center.

29. A method according to claim 27, wherein the second density is greater than the first density, and wherein orienting the eye at the intermediate downward gaze positions the optical axis to extend through the secondary fluid at the anterior optical center and the primary fluid at the posterior optical center.
